(19) United States
(12) Patent Application Publication
(10) Pub. No.: US 2026/0354688 A1
(43) Pub. Date: Nov. 12, 2026

(54) CEREBRAL NEAR INFRARED SPECTROSCOPY PROBE IN INFANTS AND PRETERM NEONATES

(71) Applicants: University of Washington, Seattle, WA (US); Seattle Children's Hospital d/b/a Seattle Children's Research Institute, Seattle, WA (US)

(72) Inventors: Sarah Kolnik, Seattle, WA (US); Ulrike Mietzsch, Seattle, WA (US); Pierre D. Mourad, Seattle, WA (US); Thomas Wood, Seattle, WA (US)

(73) Assignees: University of Washington, Seattle, WA (US); Seattle Children's Hospital d/b/a Seattle Children's Research Institute, Seattle, WA (US)

(21) Appl. No.: 19/134,586

(22) PCT Filed: Nov. 30, 2023
(86) PCT No.: PCT/US2023/081960

§ 371 Date: May 30, 2025

Related U.S. Application Data
(60) Provisional application No. 63/429,041, filed on Nov. 30, 2022.

(51) Int. Cl. A61B 5/00; A61B 5/0205; A61B 5/021; A61B 5/1455

(52) U.S. Cl. CPC A61B 5/0075; A61B 5/0205; A61B 5/14553; A61B 5/4064; A61B 5/6803; A61B 5/021; A61B 5/443; A61B 2503/045

(57) ABSTRACT

An example method includes identifying first data indicative of a first portion of VNIR light detected by a first detector; identifying second data indicative of a second portion of the VNIR light detected by a second detector; and identifying third data indicative of a third portion of the VNIR light detected by a third detector. Based on the first data, the second data, and the third data, a metric indicative of a state of a brain structure is determined. A signal, in various cases, is output based on the metric indicative of the state of the brain structure.

### CROSS-REFERENCE TO RELATED APPLICATION

This application is a U.S. National Phase Patent Application based on International Patent Application No. PCT/US2023/081960 filed on Nov. 30, 2023, which claims priority to U.S. Provisional App. No. 63/429,041, which was filed on Nov. 30, 2022, and is incorporated by reference herein in its entirety.

### BACKGROUND

Brain injury in term and preterm babies most commonly results from acute or chronic hypoxic ischemic insults and leads to lifelong sequelae such as cerebral palsy. Hypoxic ischemic injury affects at least 1% of all term and 40% of all preterm births (Osterman, et al. National Vital Statistics Reports 2022; vol. 70 no. 17, Yates, et al. Int J Mol Sci. 2021; 22(4):1671). Infants with other conditions such as heart disease or those undergoing surgical procedures are also at risk for hypoxia (Chawanpaiboon, et al. Lancet Glob Health 2019; 7: e37-e46, Douglas-Escobar, et al. JAMA Pediatr 2015; 169: 397-403, Zaleski, et al. J Cardiothorac Vasc Anesth 2020; 34: 489-500). The current standard of care is to look for signs and symptoms of hypoxic ischemic injury, which implies delayed recognition as pathological changes have already resulted or use existing devices which do not reflect the neuroanatomy of term and preterm babies.

Due to the serious consequences of cerebral hypoxic injury, about 30% of neonatal ICUs (hospitals that care for these at-risk newborns) deploy existing spectroscopy devices to monitor cerebral oxygenation in neonatal patients. However, these devices are not designed for neonatal patients. For example, the physical devices are often designed children and adults, and the analysis performed by the devices are designed for cerebral oxygenation monitoring in children and adults (Hunter, et al. Acta Paediatr 2018; 107: 1198-1204).

Due to the physiological discrepancy between adults and children, various existing spectroscopy devices fail to transmit light through predictable cerebral structures in children. In particular cases, existing spectroscopy devices produce erroneous cerebral oxygenation values in children, particularly neonates. Due to the absence of a reliable technology that can detect the occurrence of hypoxia as well monitor its treatment for neonatal patients, most (e.g., 70%) of NICUs do not deploy the existing device (6). This is evidenced by the low level of reliance for management at 9% and prognostication at 3% with the current state of spectroscopy technology (6, Li, et al. Lancet Reg Health West Pac 2021; 14: 100212, Zeitlin, et al. Int J Epidemiol 2020; 49: 372-386). However, were such a device to exist, it would allow detection of hypoxic events as well as monitoring their treatment.

### BRIEF DESCRIPTION OF THE DRAWINGS

Some of the drawings submitted herein are better understood in color. Applicant considers the color versions of the drawings as part of the original submission and reserves the right to present color images of the drawings in later proceedings.

FIG. 1 illustrates an example environment for monitoring one or more conditions of a subject 102 using cerebral spectroscopy.

FIG. 2 illustrates an example of an adjustable headband included in a spectroscopy device.

FIG. 3 is a diagram illustrating light transmission and detection by a spectroscopy device.

FIG. 4 illustrates an example process for monitoring the state of a cerebral structure.

FIG. 5 illustrates an example of at least one device configured to perform various functions described herein.

FIG. 6 illustrates a CONSORT diagram of screened and final included infants.

FIG. 7 illustrates an NIRS sensor measurement by head circumference and gestational age. FIG. 7 illustrates a Simulation of rScO₂ detection depths from the midline forehead overlying axial T2 fetal brain MRIs for increasing gestational ages as depicted in 22, 24, 26, 28, 30, 32, and 34 weeks of gestation. The non-starred and starred arches represent the underlying areas captured by the short-and long-distance emitted infrared rays, respectively.

FIGS. 8A to 8F illustrate head circumference and brain region captured by rScO2 at 2.5 cm depth from midline forehead for increasing gestational ages (22-38 weeks gestation) as derived from fetal brain MRI images. For each head circumference, the diameter (head circumference/TT) is calculated for an assumed perfectly circular head shape and the radius (diameter/2) as well as the estimated brain structure in the presumed area. FIG. 8A illustrates rScO₂ trajectories by head circumference (HC) stratified into numerical tertiles: 18-21.9 (bottom trend line), 22-25.9 (middle trend line), and 26-30 cm (top trend line). Larger HC demonstrates higher rScO₂ trajectories. FIG. 8B illustrates rSCO₂ trajectories by gestational age at birth stratified into numerical quartiles: 22-24 (bottom trend line), 25-27 (lower middle trend line), 27-30 (upper middle trend line), and >30 (top trend line) weeks of gestation. More mature infants show higher rScO₂ trajectories. FIG. 8C illustrates rScO₂ trajectories of infants with HC<26 cm stratified by gestational ages into tertiles: 22-24 (bottom trend line), 25-27 (middle trend line), and 27-30 (top trend line) weeks of gestation. FIG. 8D illustrates Cerebral NIRS rScO₂ trajectories of infants with HC≥26 cm stratified by gestational ages in tertiles: 25-27 (bottom trending to top line), 27-30 (middle trending to bottom line), and >30 (top trending to middle line) weeks gestation. FIG. 8E illustrates rScO₂ at 26 weeks GA is higher in the neonate with larger HC. The starred line represents a head circumference of 22 cm and the non-starred line represents a head circumference of 26.5 cm. FIG. 8F illustrates ScO₂ at 28 weeks remains higher in the neonate with the larger HC compared to smaller HC. The starred line represents a head circumference of 23.5 cm and the non-starred line represents a head circumference of 26 cm. Trajectories were plotted using the locally estimated scatterplot smoothing (loess) method.

FIG. 9 illustrates secondary characteristics that have been shown to influence rScO₂ measurements. FIG. 9 also illustrates theoretical interrelated factors that affect cerebral NIRS measurements in the study described in the Experimental Example. Dashed lines depict a negative relationship. Gray lines depict a positive relationship. Black lines depicts a positive or negative relationship depending on where NIRS probe is capturing signal.

FIG. 10 illustrates the different cerebral physiology of infants at different gestational ages.

FIG. 11 illustrates an example of the propagation patterns for a spectroscopy device that is suitable for infants at 25- and 37-week gestational ages.

### DETAILED DESCRIPTION

Cerebral near-infrared spectroscopy (cNIRS) is currently widely used with a system that was designed and configured for adult and pediatric patients. Large discrepancies exist between adult, pediatric, and neonatal head circumferences and tissue composition. A small head size alters the depth of the signal and evolving tissue composition alters the optical path of the NIRS signal. Previous systems output NIRS values which are extrapolated from algorithms designed for adult and pediatric patients, which produce inaccurate measurements for infant and preterm patients.

Various implementations described herein relate to devices, systems, and methods for performing cNIRS on different types of subjects, such as neonatal and/or preterm subjects. According to some examples, a noninvasive spectroscopy device emits near-infrared (NIR) light into cranial tissue of a subject. The device includes at least three detectors configured to detect the NIR light scattered from various types of cranial tissue. In some examples, at least one of the detectors receives NIR light scattered from superficial tissues, such as the skin, skull, muscle, or any combination thereof. In some cases, at least one of the detectors receives NIR light scattered from brain tissue and vasculature traversing the brain tissue. In some examples, at least one of the detectors receives NIR light scattered from one or more ventricles of the subject.

The amount of NIR scatter received by the detectors is dependent on the amount of blood in the tissues from which the NIR light is scattered. For instance, blood absorbs and/or scatters NIR light differently than other physiological tissues (e.g., brain tissue) and fluids (e.g., cerebrospinal fluid (CSF)). Further, deoxygenated blood absorbs and/or scatters NIR light differently than oxygenated blood. Based on the NIR scatter detected by the detectors, the device may monitor a condition of the subject. For instance, the device may infer whether the subject is experiencing an interventricular hemorrhage based on the scatter from the ventricle(s). In cases where oxygenated blood is entering the ventricles, the blood may increase absorbance of the light propagating through the ventricle(s). In cases where blood in the ventricles begins to clot, the blood may lose oxygenation, thereby reducing the absorbance of the light propagating through the ventricle(s). In various cases, a rapid change (with respect to time) in absorbance of the light propagating through the ventricle(s) may be indicative of interventricular hemorrhage. Since intraventricular hemorrhage is a significant source of hypoxic-ischemic injury in neonates, various implementations of the present disclosure can enable noninvasive monitoring of an acute health problem for this population.

In some examples, the NIR light (or light in a different spectrum) is differentially absorbed by bilirubin. According to various cases, the amount of scatter received by the detectors is indicative of an amount of bilirubin in the CSF of the ventricle(s). In some examples, the device outputs an indication or alert based on the amount of bilirubin in the CSF.

Previous cNIRS devices are designed with non-adjustable spacing between light sources and detectors. These devices are not suitable for neonatal subjects (e.g., preterm infants) who experience rapid growth in head size as a function of gestational age. The rapid growth in head size results in a corresponding growth in head circumference. As a result, a device with fixed spacing between light source(s) and detector(s) monitors vastly different cerebral structures at different ages.

In contrast, various implementations of the present disclosure include devices with adjustable spacing between light sources and detectors. For example, a device includes a headband with an adjustable portion that can be used to change the distance between a light source and detectors, or between different detectors, along the length of the headband. In some cases, these distances can be adjusted to enable the device to a specific cerebral region-of-interest. A user, for instance, may adjust these lengths based on gestational age, head circumference, and other characteristics of the subject being monitored. The adjustable headband therefore adapts to the rapidly growing head size and allows for precise monitoring of the intended portion of brain.

Implementations of the present disclosure provide several technical improvements to the field of cerebral monitoring. In various cases, adjustable spacings between light sources and detectors enable devices described herein to be used with different types of patient populations, with patients who experience rapid physiological changes (e.g., neonates), and the like. In some examples described herein, spectroscopy devices can be used to monitor cerebral ventricles of subjects, thereby enabling rapid and accurate detection of intraventricular hemorrhage and other ventricle-related conditions.

Various implementations of the present disclosure will now be described with reference to the accompanying figures.

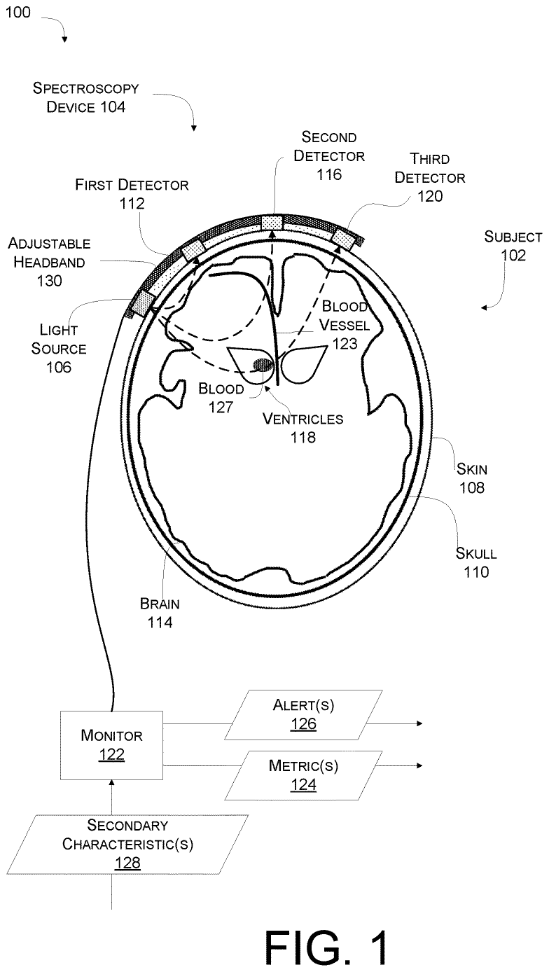

FIG. 1 illustrates an example environment 100 for monitoring one or more conditions of a subject 102 using cerebral spectroscopy. In various cases, the subject 102 is a human. For example, the subject 102 is a neonate. The terms “neonate,” “neonatal subject,” and their equivalents, may refer to an infant that is younger than four weeks. In some cases, the subject 102 is younger than 72 hours. In various cases, the subject 102 is a preterm infant. The term “preterm infant,” and its equivalents, may refer to a baby that is born alive prior to a full gestational term (37 weeks for humans).

In particular cases, the subject 102 is in a continuous monitoring environment, such as an intensive care unit (ICU), pediatric ICU (PICU), neonatal ICU (NICU), or the like. For example, the subject 102 may have an unstable condition and may be prone to one or more serious medical problems. Various physiological parameters of the subject 102 may be discretely and manually monitored by a care provider, such as a nurse or physician. As used herein, the term “physiological parameter,” and its equivalents, may refer to a metric that is indicative of a physical condition of a subject. Examples of physiological parameters, for instance, include vital signs (e.g., pulse rate, heart rate, core temperature, respiration rate, blood pressure, etc.), ventilation parameters (e.g., respiration rate, respiration volume, ventilation rate, ventilation volume, partial pressure of CO₂ or O₂ in respired air, air pressure in the lungs, flow rate, end-tidal parameters, such as end-tidal CO₂, etc.), cardiac parameters (e.g., electrocardiogram (ECG), heart rate, etc.), blood parameters (e.g., hematocrit, blood gas measurements, the presence or amount of one or more molecules (e.g., glucose) in the blood, blood pressure, blood flow, pulse oximetry, regional blood oxygenation, etc.), neural parameters (e.g., electroencephalogram (EEG), intracranial pressure measurements, cerebral oxygenation, etc.), renal parameters (e.g., urine output), and the like. A physiological parameter is monitored “continuously,” for instance, if it is measured or calculated repeatedly and/or periodically (e.g., once every second, minute, hour, etc.). In various cases, a sensor may sample a physiological parameter continuously without being prompted by a user at each measurement. For example, a care provider may attach a blood pressure sensor to the subject 102 and may set the blood pressure sensor to measure the blood pressure of the subject 102 at a predetermined frequency (e.g., once every hour). The blood pressure sensor may perform the specified measurements at the predetermined frequency without further interaction between the blood pressure sensor and the care provider. In some examples, continuous monitoring may increase the chance that a change in the condition of the subject 102 may be identified quickly and can be addressed effectively.

Cerebral monitoring is of particular interest, particularly in neonatal subjects. For example, the subject 102 may be prone to a condition that reduces blood flow to the brain 114 of the subject. If the brain 114 receives insufficient blood flow for an extended period of time, the subject 102 may experience serious and/or irreversible damage to the brain 114. It may therefore be advantageous to monitor an amount of blood (e.g., oxygenated blood) flowing through the brain 114 of the subject 102.

A spectroscopy device 104 is configured to monitor the brain 114 of the subject 102. In particular, the spectroscopy device 104 may be configured to optically monitor cerebral oxygenation of the subject 102. Cerebral oxygenation, in various cases, is a measurement of regional blood oxygenation of blood traversing the brain 114. In various implementations, the spectroscopy device 104 is configured to measure cerebral oxygenation of the subject 102 repeatedly and/or periodically.

In various cases, the spectroscopy device 104 includes a light source 106 configured to emit electromagnetic radiation into the head of the subject 102. In various examples, the electromagnetic radiation includes light. The light emitted by the light source 106, in various cases, includes visible light, near infrared (NIR) light, infrared (IR) light, or any combination thereof. For instance, the light source 106 may emit visible and NIR (VNIR) light. The light emitted by the light source 106, for instance, includes one or more wavelengths in a range of 620 nanometers (nm) to 200 micrometers (μm) or in a range of 800 nm to 2.5 μm. The light source 106, for instance, includes one or more light-emitting diodes (LEDs), one or more incandescent bulbs, one or more quarts halogen bulbs, or any combination thereof.

Due to the wavelength(s) of the light emitted by the light source 106, the light emitted by the light source 106 is propagated through, absorbed, and scattered by various structures within the head of the subject 102. As used herein, the terms “structure,” “physiological structure,” and their equivalents, may refer to tissues, fluids, spaces, bone, or other physiological elements of a subject, such as the subject 102. As used herein, the term “scatter,” and its equivalents, refers to a process by which at least one particle, or a boundary between different mediums, receives electromagnetic radiation and changes the direction and/or amplitude of the electromagnetic radiation. In some examples, light that is scattered from a particle or boundary is also referred to as “scatter.” In various cases, light can be absorbed by materials, thereby reducing the intensity (e.g., amplitude) of the light scattered and otherwise transmitted from those materials.

In particular, at least a portion of the light emitted from the light source 106 is scattered by superficial structures on the head of the subject 102, such as skin 108 and the skull 110 of the subject. In various implementations, the superficial structures also include other tissues, such as dura and muscle, disposed between the skin 108 and skull 110 of the subject 102. Due to the optical characteristics of the superficial structures, and the relatively short depth of the superficial structures in the head of the subject 102, the light scattered by the superficial structures forms an arc with a relatively small radius. In various cases, the superficial structures may be located at a depth of 1 centimeter (cm) to 2 cm below the surface of the head of the subject 102. The light scattered by the superficial structures, for instance, is received by a first detector 112 that is located relatively close to the light source 106. According to some examples, a distance between the light source 106 and the first detector 112 (e.g., along the surface of the head of the subject 102) is in a range of 1 cm to 8 cm. In some cases, the spectroscopy device 104 is configured to detect the state of one or more non-cerebral structures. For instance, the light emitted from the light source 106 may be scattered by superficial structures (e.g., skin, muscle, bone, etc.) on other parts of the body of the subject 102.

In addition, at least a portion of the light emitted from the light source 106 is propagated through the superficial structures, thereby penetrating deeper structures in the head of the subject 102. In various cases, the light emitted from the light source 106 propagates through and is scattered by the brain 114 of the subject 102. In various implementations, the brain 114 is located at a deeper depth from the surface of the head than the superficial structures. Accordingly, the light scattered by the brain 114 forms an arc with a longer radius than the light scattered by the superficial structures. The light scattered by the superficial structures is therefore received by a second detector 116. In various cases, the first detector 112 is disposed between the light source 106 and the second detector 116. According to various examples, the second detector 116 is separated from the light source 106 by a greater distance than the distance between the light source 106 and the first detector 112. In various cases, the light detected by the second detector 116 may be emitted from a portion of the brain 114 that is located a depth in a range of 1 cm to 8 cm below the surface of the head of the subject 102. In some cases, a distance between the first detector 112 and the second detector 116 (e.g., along the surface of the head of the subject 102) is in a range of 0.5 cm to 2.5 cm.

In various examples, at least a portion of the light emitted from the light source 106 is transmitted through the superficial structures and through at least a portion of the brain 114 of the subject 102. For example, in cases wherein the subject 102 is a neonate, the relative size of the head of the subject 102 and the optical properties of structures in the head of the subject 102 enable transmission of the light from the light source 106 to deep structures within the head of the subject 102. In particular cases, the light from the light source 106 is transmitted into one or more ventricles 118 of the brain 114 of the subject 102. The ventricles 118, for instance, are cavities located within the parenchyma of the brain 114. The ventricles 118, in various cases, contain cerebrospinal fluid (CSF). The ventricles 118 are located at a relatively deep depth in the head of the subject 102. Accordingly, the light that is propagated through and scattered from the ventricles 118 may form an arc that has a relatively large radius. At least a portion of the light scattered from the ventricles 118 is received by a third detector 120, for instance. A distance between the third detector 120 and the light source 106 is greater than the distance between the light source 106 and the first detector 112 or the distance between the light source and the second detector 116. In various cases, the third detector 120 detects light emitted from a structure that is disposed below the surface of the head of the subject 102 by a depth in a range of 1.5 cm to 10 cm. For instance, a distance between the second detector 116 and the third detector 120 along the spectroscopy device 104 may be in a range of 0.5 cm to 2.5 cm.

The first detector 112, the second detector 116, and the third detector 120 are each configured to detect light. For example, the first detector 112, the second detector 116, and the third detector 120 are each configured to detect an intensity of the light that they receive. In various examples, the first detector 112, the second detector 116, and the third detector 120 are configured to detect light having wavelength(s) that is consistent with the light emitted by the light source 106. Accordingly, the intensities detected by the first detector 112, the second detector 116, and the third detector 120 are indicative of an amount of the light that is transmitted by the light source 106 and through various structures in the head of the subject 102. For example, the first detector 112, the second detector 116, and the third detector 120 may include at least one photodiode (e.g., a P-N photodiode, a P-I-N photodiode, an avalanche photodiode) at least one metal-semiconductor-metal (MSM) photodetector, at least one charge-coupled device (CCD), at least one complementary metal oxide semiconductor (CMOS) sensor, at least one photomultiplier tube (PMT), or any combination thereof.

In various examples, the spectroscopy device 104 outputs one or more signals indicative of the light detected by the first detector 112, the second detector 116, and the third detector 120 to a monitor 122. The monitor 122 may be an external device that is communicatively coupled to the spectroscopy device 104. Although not specifically illustrated in FIG. 1, in some cases, the monitor 122 is part of the spectroscopy device 104. In various implementations, the first detector 112, the second detector 116, and the third detector 120 generate analog signals indicative of the respective amount of light they detect. In some examples, the spectroscopy device 104 includes at least one analog-to-digital converter (ADC) configured to generate a digital signal based on the analog signals. In some cases, the first detector 112, the second detector 116, and the third detector 120 respectively generate analog signals indicative of the respective amount of light they detect. The signals output by the spectroscopy device 104 to the monitor 122 include at least one of the digital signals and/or at least one of the analog signals.

In various cases, the monitor 122 is configured to analyze the light detected by the first detector 112, the second detector 116, and the third detector 120. The amount of light emitted by the light source 106 that is detected by the first detector 112, the second detector 116, and the third detector 12, is indicative of one or more physiological conditions of the subject 102. In particular cases, oxygenated blood has a different absorbance of the spectra of the light emitted by the light source 106 than other physiological structures (e.g., brain tissue, deoxygenated blood, bone, CSF, etc.). In various cases, when hemoglobin is bound to oxygen, the hemoglobin absorbs light having the spectra of the light emitted by the light source 106. In various examples, the physical characteristics of the cerebral structures along the arcs of light emitted by the light source 106 and received by the first detector 112, the second detector 116, and the third detector 120 impact the amount of light detected by the first detector 112, the second detector 116, and the third detector 120. For instance, an increase in the amount of oxygenated blood along the arc between the light source 106 and the third detector 120 may reduce the amount of the light detected by the third detector 120, due to the oxygenated blood scattering (e.g., absorbing) of at least a portion of the light emitted from the light source 106. Thus, the amount of light that is transmitted through the head of the subject 102 by the light source 106, and is detected by the first detector 112, the second detector 116, and the third detector 120, can be indicative of one or more cerebral conditions of the subject 102.

In various cases, oxygenated blood is not the only type of material in the head of the subject 102 that scatters (e.g., absorbs) at least a portion the light emitted by the light source 106. For instance, the skin 108, skull 110, brain 114, deoxygenated blood, and CSF may also absorb at least some of the light emitted by the light source 106. In various cases, the amount of light transmitted through these structures (and detected by the first detector 112, the second detector 116, and the third detector 120) depends on the developmental state of the subject 102 and/or physiology of the subject 102.

According to various instances, the monitor 122 determines a condition of the superficial tissues of the head of the subject 102 by analyzing the amount of light detected by the first detector 112. For instance, the monitor 122 may compare an amount of light emitted by the light source 106 to an amount of light detected by the first detector 112. Based on the comparison, the monitor 122 may determine the presence of oxygenated blood within the path of the light between the light source 106 and the first detector 112.

In various examples, the monitor 122 determines a condition of the brain 114 of the subject 102 by analyzing the amount of light detected by the first detector 112 and the amount of light detected by the second detector 116. In particular cases, the light detected by the second detector 116 travels through the superficial tissues of the head of the subject 102 in addition to the brain 114 tissue of the subject 102. The light detected by the first detector 112 travels primarily through the superficial tissues of the head of the subject 102 while traveling less through brain 114 tissue of the subject 102. Accordingly, a discrepancy between the light at a given wavelength (e.g., a red wavelength, an NIR wavelength, etc.) detected by the first detector 112 and the light detected by the second detector 116 may be indicative of an amount of oxygenated blood in the brain 114 tissue of the subject along the transmission path of the light detected by the second detector 116. For instance, the discrepancy can be represented by the following Equation 1:

A_{B}=I_{D2}−a₁*I_{D1} (1)

wherein A_{B} is a metric proportional to a net signal that arrives at the spectroscopy device 104 that has propagated primarily through the brain 114 tissue, I_{D1} is an amount (e.g., magnitude) of light detected by the first detector 112, and ID₂ is an amount (e.g., magnitude) of light detected by the second detector 116 and the coefficient a₁ alters the relative weights of the signal at a given wavelength, and can be derived based upon empirical data.

For instance, the light traveling between the light source 106 and the second detector 116 passes through and/or is scattered by a blood vessel 123 in the brain 114 of the subject 102. The blood vessel 123, in various cases, is a physiological structure that carries blood through the brain 114 of the subject 102. The blood vessel 123, for instance, includes an artery or a vein. Thus, in various cases, the discrepancy between the light detected by the first detector 112 and the light detected by the second detector 116 is indicative of an amount of oxygenated blood traveling through the blood vessel 123.

In some examples, the amount of light absorbed by the brain 114 tissue between the superficial tissue (hair, skin, fat, fascia, muscle, etc.) and the deep portion of the brain 114 that contains the ventricles 118, can be determined based on the following Equation 2:

A_{B}=I_{D2}−a₂*I_{D1}−a₃*I_{D3} (2)

wherein I_{D3} is an amount (e.g., magnitude) of light detected by the third detector 120, a₂ is a coefficient that alters the relative weights of the signal at the given wavelength (e.g., derived upon empirical data), and a₃ is a coefficient that alters the relative weights of the signal at the given wavelength (e.g., derived upon empirical data).

In various implementations, the monitor 122 outputs one or more metrics 124 that indicate the condition of the superficial structures and/or the brain 114 of the subject 102. For example, the metric(s) 124 may include a value that is proportional to, or otherwise dependent on, the amount of light absorbed by one or more cerebral structures (e.g., the brain 114) of the subject 102. For example, the monitor 122 may calculate a cerebral oxygenation of the subject 102 using the following Equation 3:

O_{C}=f_{C}*A_{B} (3)

Wherein O_{C} is a cerebral oxygenation of the subject 102 and f_{C} is a scaling factor. In various cases, f_{C} is dependent on the physiology of the subject 102. In some cases, O_{C} is a percentage representing [(oxygehemoglobin/oxyhemoglobin+deoxyhemoglobin)*100] of the blood in the brain 114 of the subject 102. O_{C} is one example of the metric(s) 124.

In some cases, the metric(s) 124 include a rate of change of the amount of light transmitted through one or more cerebral structures (e.g., at least a portion of the brain 114) of the subject 102, or a metric related to the amount of light transmitted through the cerebral structure(s). For example, the metric(s) 124 may include a value of a derivative of A_{B} or O_{C} with respect to time.

Further, in some cases, the monitor 122 generates and outputs one or more alerts 126 based on the condition of the superficial structures and/or the brain 114 of the subject 102. In some cases, the monitor 122 compares a metric indicative of cerebral oxygenation (e.g., a metric dependent on the amount of light absorbed by at least a portion of the brain 114 including one or more blood vessels) to a lower threshold and an upper threshold. If the metric is below the lower threshold, or if the metric is above the upper threshold, the monitor 122 may output the alert(s) 126. In various cases, low absorbance of NIR light by the brain 114 could indicate that there is insufficient oxygenated blood in the brain 114. In some examples, the monitor 122 compares a metric indicative of a rate of change of cerebral oxygenation (e.g., the rate of change of the amount of light absorbed by the brain 114 with respect to time) to a lower threshold and an upper threshold, and outputs the alert(s) 126 in response to determining that the rate of change is below the lower threshold or above the upper threshold.

In various implementations, the monitor 122 is configured to output signals that are indicative of a state of the ventricles 118. Thus, the spectroscopy device 104 and/or monitor 122 can enable a care provider utilizing the monitor 122 to be notified of problems with the ventricles 118. In some cases, blood 127 may leak into the ventricles 118 from at least one blood vessel 123 in the brain 114. For example, the blood 127 may leak from the blood vessel 123 into the ventricles 118. In various cases, the presence of the blood 127 is indicative of an intraventricular hemorrhage. In some examples, the blood 127 may increase a fluid pressure within the ventricles 118, which may physically damage cells in the brain 114. In some cases, intraventricular hemorrhage can lead to irreversible brain damage. Moreover, symptoms of intraventricular hemorrhage, such as bradycardia and apnea, are also indicative of other serious conditions. Thus, it is highly clinically valuable to be able to directly detect the presence and/or amount of the blood 127 in the ventricles 118.

According to some implementations, the monitor 122 determines a condition of the ventricles 118 of the subject 102 by analyzing the amount of light detected by the first detector 112, the amount of light detected by the second detector 116, and the amount of light detected by the third detector 120. For example, at least a portion of the light detected by the third detector 120 is transmitted through the ventricles(s) 118, however, at least a portion of the light detected by the first detector 112 and the second detector 116 is not transmitted through the ventricles 118. The amount of oxygenated blood in the ventricles 118 can be determined by isolating the amount of light that as propagated through the ventricles 118 from the light that has propagated through the superficial tissues and the brain 114 tissue itself. One may estimate this quantity using the following Equation 4:

A_{V}=I_{D3}−a₄*I_{D1}−a₅*I_{D2} (4)

wherein Aᵥ a metric proportional to a net signal that arrives at the spectroscopy device 104 that has propagated primarily through the ventricles 118 tissue with the contributions of the superficial tissues and the brain 114 removed, a₄ is a coefficient that alters the relative weights of the signal at the given wavelength (e.g., derived upon empirical data), and as is another coefficient that alters the relative weights of the signal at the given wavelength (e.g., derived upon empirical data).

According to some examples, the monitor 122 calculates a blood oxygenation representative of the ventricles 118 according to the following Equation 5:

O_{V}=f_{V}*A_{V} (5)

wherein O_{V} is a metric related to the net propagation of the light through the portion of the brain 114 containing the ventricles 118 (with the contribution of the superficial layers and brain 114 tissue removed) and is proportional to an amount of oxygenated blood in the ventricles 118 and f_{V} is a scaling factor. In various cases, O_{V} is representative of the presence and/or amount of the blood 127 in the ventricles 118 and can therefore be utilized to assess whether the subject 102 is experiencing ventricular hemorrhage. In some examples, f_{V} is dependent on the physiology of the subject 102. Aᵥ and O_{V} are examples of the metric(s) 124.

In various examples, the metric(s) 124 and/or alert(s) 126 are generated by the monitor 122 based on the condition of the ventricles 118. For example, in some cases, the metric(s) 124 include the ventricle absorbance (A_{V}) and/or a value calculated based on the ventricle absorbance (e.g., O_{V}). In some cases, the metric(s) 124 may include derivative of the A_{V} or O_{V} with respect to time. In some instances, the monitor 122 compares the metric(s) 124 to a lower threshold and an upper threshold. The monitor 122 may generate and output the alert(s) 126 in response to determining that the metric(s) 124 are below the lower threshold or above the upper threshold. For instance, a decrease in an amount of VNIR light that has propagated through the ventricle(s) 118 may indicate that the blood 127 is oxygenated and leaking into the ventricle(s) 118.

In some cases, the subject 102 may have specific physiology that impacts the transmission path of the light emitted by the light source 106, the scatter of the light, and the amount of light absorbed by structures within the head of the subject 102. In particular cases, the size of the head of the subject 102 impacts the light detected by the first detector 112, the second detector 116, and the third detector 120. For example, the head circumference of the subject 102 may impact the depth of the brain 114 and ventricles 118 with respect to the surface of the skin 108 on which the light source 106 and the first detector 112, second detector 116, and third detector 120 are disposed. In particular cases, the ventricles 118 of preterm neonates are located at a relatively shallower cerebral depth as compared to adult patients.

As described in further detail below with respect to the Experimental Example, several other factors impact a relationship between the cerebral oxygenation and ventricle state of the subject 102 and the light detected by the first detector 112, the second detector 116, and the third detector 120. The developmental stage of the subject 102, for instance, may impact the light that is detected by the first detector 112, the second detector 116, and the third detector 120. For example, preterm infants may have different skin thickness, skull thickness, ventricular size, vasculature, or brain composition (e.g., composition of white versus gray matter) than other patient populations (e.g., term infants, pediatric patients, adults, etc.). The different composition of cerebral structures in preterm infants may impact the amount of light scattered by the cerebral structures.

Other aspects of the physiology of the subject 102 may impact the metric(s) 124. For example, a pigmentation of the skin 108 of the subject 102 may impact how much of the light is absorbed or scattered by the skin 108. In some cases, a blood pressure of the subject 102 (e.g., indicative of a pressure in the blood vessel 123) impacts how much of the light is absorbed or scattered in cerebral tissues. A composition (e.g., hematocrit, glucose level, etc.) of the blood of the subject 102, in some cases, impacts the scatter of light through cerebral tissues.

In some examples, the monitor 122 calculates the metric(s) 124 based on one or more secondary characteristics 128 of the subject 102. The secondary characteristics 128, for instance, include additional biomarkers of the subject 102, such as physiological parameters, demographic information, and the like. Examples of the secondary characteristic(s) 128 include at least one of an age of the subject 102 (e.g., a gestational age or age since birth); a weight of the subject 102 (e.g., a birth weight, current weight, or body mass index (BMI)); a head circumference of the subject 102 (e.g., head circumference at birth or current head circumference); a skin pigmentation of the subject 102; a bilirubin state of the subject 102 (e.g., a jaundice severity); an oxygen saturation of the subject 102 (e.g., a pulse oxygenation or regional oxygenation measured on a non-head portion of the body); a partial pressure of carbon dioxide (pCO₂) in blood of the subject 102; a blood pressure of the subject 102 (e.g., diastolic or systolic blood pressure); a skin thickness of the subject 102 (e.g., a thickness of the skin 108); a skull thickness of the subject 102; or a hematocrit of the subject 102. In various cases, the monitor 122 utilizes the secondary characteristic(s) 128 to estimate cerebral oxygenation and/or a state of the ventricles 118 based on the light absorbed by the first detector 112, the second detector 116, the third detector 120, or a combination thereof. For example, the monitor 122 may calculate f_{C} and/or f_{V} based on the secondary characteristic(s) 128.

In various examples, the size and/or shape of the head of the subject 102 may be relatively small or may change rapidly over time. For example, neonatal head circumference changes significantly in the weeks after birth. As a result, if the spacing between the light source 106, the first detector 112, the second detector 116, and the third detector 120 is constant, then the transmission arcs through the head of the subject 102 may change positions as the subject 102 ages. If the relative distance between the light source 106 and the third detector 120 remains constant, the third detector 120 may detect light transmitted through the ventricles 118 when the subject 102 is one day old but may detect light that is not transmitted to the ventricles 118 when the subject 102 is ten days old.

According to various implementations of the present disclosure, the spectroscopy device 104 includes an adjustable headband 130 that enables adjustment between the spacing of the light source 106, the first detector 112, the second detector 116, and the third detector 120. In various cases, the light source 106, the first detector 112, the second detector 116, and the third detector 120 are disposed on and/or inside of the adjustable headband 130. In some examples, the adjustable headband 130 is adhered to the skin 108 of the subject 102, such as via a biocompatible adhesive. The biocompatible adhesive, in various implementations, is transmissive to the light emitted by the light source 106. In various examples, the biocompatible adhesive is gentle on the skin 108 of the subject 102, particularly in cases wherein the subject 102 is a neonate and the skin 108 is relatively fragile. In some cases, the adjustable headband 130 is strapped around the head of the subject 102, such as by a headband.

According to some examples, the adjustable headband 130 may have at least one adjustable length. In various cases, the adjustable headband 130 includes an elastic and/or compressive material that can be lengthened if subjected to a tension or shortened if subjected to a compressive force. For example, the adjustable headband 130 may include a silicone material, polyvinyl chloride (PVC), polyester, cotton, urethane, or nylon. Although FIG. 1 illustrates that the adjustable headband 130 is disposed on portion of a circumference of the head of the subject 102, implementations are not so limited. For example, ends of the adjustable headband 130 may be configured to be coupled together (e.g., by a buckle or other type of fastener), such that the adjustable headband 130 extends around a circumference of the head of the subject 102.

In some examples, the adjustable headband 130 may include at least one adjustment mechanism that can be used to increase and/or decrease at least one length of the adjustable headband 130. For example, the adjustable headband 130 may include a first portion coupled to a buckle, pin, or hook, and a second portion that includes multiple holes in which the buckle, pin, or hook is configured to be inserted. The length of the first portion and the second portion may be based on the hole of the second portion in which the buckle, pin, or hook is inserted. In some examples, the adjustable headband 130 includes a hook-and-loop fastener (e.g., a VELCRO™ fastener) that can be used to adjust a length of the adjustable headband 130.

One or more portions of the adjustable headband 130 may have adjustable lengths. For example, a portion of the adjustable headband 130 disposed between the light source 106 and the first detector 112, a portion of the adjustable headband 130 between the first detector 112 and the second detector 116, a portion of the adjustable headband 130 between the second detector 116 and the third detector, or any combination thereof, may have an adjustable length.

The adjustable headband 130, in various cases, enables the spectroscopy device 104 to be utilized with different subjects with different physical characteristics. For example, the adjustable headband 130 may enable the spectroscopy device 104 to monitor cerebral oxygenation and ventricular state of the subject 102, as well as another subject with a different head circumference, age, and cerebral composition. For example, the length of the portion of the adjustable headband 130 between the light source 106 and the first detector 112 may be shortened, which may enable the spectroscopy device 104 to be used to monitor a younger subject with a shorter head circumference. In some examples, the adjustable headband 130 enables the spectroscopy device 104 to be utilized by the subject 102 as the subject 102 grows or otherwise matures. For example, the length of the adjustable headband 130 disposed between the light source 106 and the first detector 112 may be increased as the head circumference of the subject 102 increases, such that the second detector 115 may consistently receive light traveling through the brain 114 of the subject 102 and that the third detector 120 may consistently receive light traveling through the ventricles 118 of the subject 102 as the subject 102 ages.

In some implementations, the adjustable headband 130 otherwise enables the first detector 112 to be positioned such that it receives an arc of light emitted by the light source 106 that travels through the superficial tissues of the head of the subject 102; enables the second detector 116 to be positioned such that it receives an arc of light emitted by the light source 106 that travels through the brain 114 and blood vessel 123 of the subject 102; and enables the third detector 120 to be positioned such that it receives an arc of light emitted by the light source 106 that travels through the ventricles 118 of the subject 102. In some cases, the adjustable headband 130 includes a flexible material, such as a flexible plastic sheet or cloth. In various examples, an adhesive is disposed on the flexible material in a location that is adjacent to (e.g., within a threshold distance, such as 10 millimeters (mm) of) the first detector 112 and is disposed on the flexible material in a location that is adjacent to the light source 106. A user may manually adjust the distance between the light source 106 and the first detector 112 along the circumference of the head of the subject 102 by independently adhering the two portions of adhesive on the skin 108 of the subject. Optionally, the portion of the adjustable headband 130 disposed between the light source 106 and the first detector 112 may be folded or otherwise separated from the skin of the subject 102, such that the distance between the light source 106 and the first detector 112 along the skin 108 of the subject may be shorter than a distance between the light source 106 and the first detector 112 along the adjustable headband 130.

In some examples, the environment 100 instructs a user (e.g., a care provider) how to set a length of the adjustable headband 130 that is appropriate for the subject 102. For example, guidelines can be printed on the adjustable headband 130 that indicate an appropriate length of the adjustable headband 130 by head circumference, gestational age, or the like. In some cases, the monitor 122 instructs the user on setting a length of the adjustable headband 130. For instance, the monitor 122 may analyze the secondary characteristics 128 in order to identify an appropriate length of the adjustable headband 130 for the subject 102 and may report the appropriate length of the adjustable headband 130 to the user.

Although not specifically illustrated in FIG. 1, in some cases, the spectroscopy device 104 includes, or is communicatively coupled to, at least one additional sensor configured to detect an additional physiological parameter of the subject 102. In some cases, the additional sensor is disposed on, or otherwise integrated with, the adjustable headband 130, thereby efficiently utilizing space on the skin 108 of the subject 102 for physiological monitoring. The spectroscopy device 104 and/or the monitor may be communicatively coupled to ECG electrodes configured to detect an ECG of the subject 102, EEG electrodes configured to detect an EEG of the subject 102, a blood glucose monitor configured to detect a level of glucose in the blood of the subject 102, a hematocrit sensor configured to detect a hematocrit of the subject 102, a medical imaging device (e.g., a device including an ultrasound transducer) configured to detect a skin thickness of the subject 102, a thermometer configured to detect a temperature of the subject 102, a blood pressure cuff configured to detect a blood pressure of the subject 102, or any combination thereof. In some cases, the additional sensor is configured to detect at least one of the secondary characteristic(s) 128. In some examples, the physiological parameter(s) detected by the additional sensor is independently analyzed and/or output by the monitor 122.

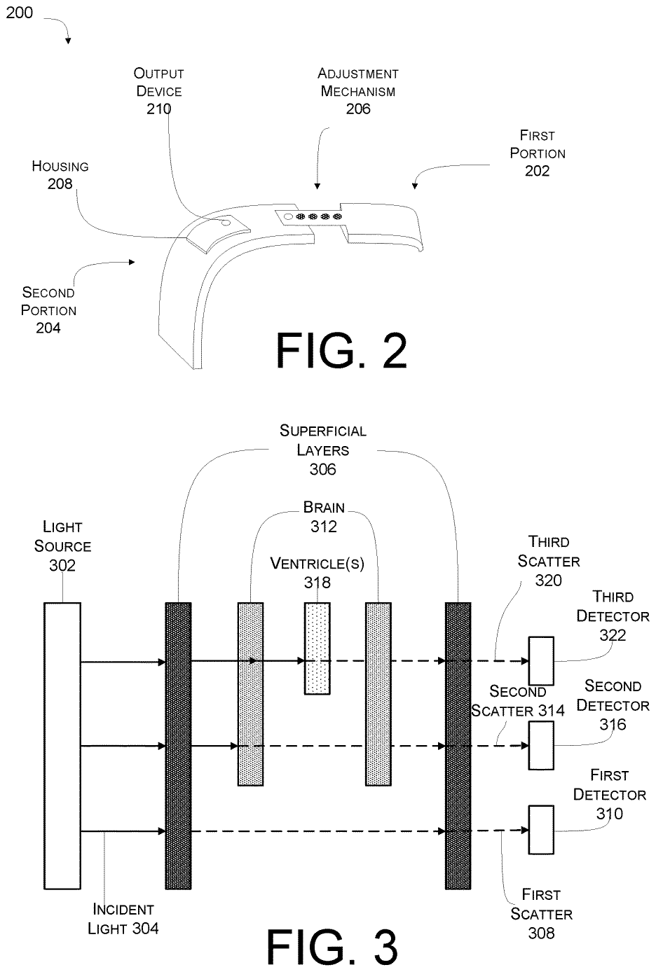

FIG. 2 illustrates an example of an adjustable headband 200 included in a spectroscopy device. In some examples, the adjustable headband 200 is the adjustable headband 130 described above with reference to FIG. 1.

The adjustable headband 200 includes a first portion 202 and a second portion 204. Although not specifically illustrated in FIG. 2, the first portion 202 may include one or more light emitters and/or one or more light detectors. The second portion 204, in various cases, includes one or more light sources and/or one or more light detectors. For instance, the first portion 202 may include the light source 106 and the second portion 204 may include the first detector 112, the second detector 116, and the third detector 120. In various cases, the light source(s) and/or light detector(s) are disposed on a bottom surface of the adjustable headband 200, such that they are not visible from the perspective illustrated in FIG. 2.

In various cases, the first portion 202 and the second portion 204 include a flexible material. Thus, the first portion 202 and the second portion 204 may conform to a curved surface, such as a portion of a head of a subject. In some cases, the flexible material includes a flexible polymer sheet (e.g., a silicone material, polyvinyl chloride (PVC), or the like). In some examples, the flexible material includes a woven or webbed material, such as a polyester, cotton, urethane, or nylon material. In some cases, at least a portion of the bottom surface of the adjustable headband 200 is coated with a biocompatible adhesive, such as an acrylate-, silicone-, polyurethane-, hydrocolloid-, or hydrogel-containing adhesive.

The first portion 202 and the second portion 204 are coupled together via an adjustment mechanism 206. The adjustment mechanism 206, for instance, includes a pin extending from a top surface of the second portion 204 as well as several holes extending through the first portion 202. The adjustment mechanism 206 can be used to adjust a length of the adjustable headband 200 and to couple the first portion 202 to the second portion 204. For example, the second portion 204 is coupled to the first portion 202 when the pin extending from the second portion 204 is inserted through any of the holes of the first portion 202. The hole in which the pin is inserted is determinative, for instance, of the length of the adjustable headband 200. For instance, as illustrated in FIG. 2, the pin is inserted through a distal hole of the first portion 202, such that the length of the adjustable headband 200 is maximized. However, if the pin was inserted through a more proximal hole of the first portion 202, the length of the adjustable headband 200 could optionally be shortened. In various cases, the adjustable length of the adjustable headband 200 enables an adjustable distance between at least one light emitter and at least one light detector disposed on the adjustable headband 200.

In various cases, a housing 208 is disposed on a top surface of the adjustable headband 200. In various cases, the housing 208 at least partially encloses a circuit and/or processor that is communicatively coupled with the light source(s) and light detector(s) disposed on the adjustable headband 200. In some cases, the housing 208 further encloses a power source (e.g., a battery) that is configured to supply a voltage to the circuit. The circuit and/or processor, in various cases, may be configured to adjust an output of the light emitter(s), determine an amount of light received by the light detector(s), analyze the amount of light received by the light detector(s), generate signals indicative of the amount of light received by the light detector(s), transmit the signals indicative of the amount of light received by the light detector(s), or any combination thereof.

An output device 210 is disposed on, in, or otherwise integrated with the housing 208. In various cases, the output device 210 is configured to output a signal based on the amount of light received by the light detector(s). The output device 210 may include a transceiver, an LED, or the like. For example, the output device 210 may include an LED that flashes if the circuit and/or processor determines that an amount of light detected by one or more of the light detectors is indicative of bleeding in the ventricles of a subject.

FIG. 3 is a diagram illustrating light transmission and detection by a spectroscopy device. A light source 302 is configured to emit incident light 304. The incident light 304, for instance, includes NIR light. The light source 302 is part of the spectroscopy device. For example, the light source 302 is disposed on an adjustable headband that is disposed on the head of a subject.

The incident light 304 is transmitted to superficial layers 306, such as skin, muscle, and bone. At least a portion of the incident light 304 transmitted to the superficial layers 306 is scattered by the superficial layers 306, thereby generating first scatter 308. The first scatter 308, in various cases, is received by a first detector 310.

Some of the incident light 304 is transmitted through the superficial layers 306. A portion of the incident light 304 is transmitted through the superficial layers 306 and scattered by the brain 312 of the subject. For example, second scatter 314 is scattered by the brain 312 and transmitted through the superficial layers 306. The second scatter 314 is received by a second detector 316.

In various cases, a cerebral oxygenation of the subject can be determined based on the first scatter 308 and the second scatter 314. A discrepancy between an amount of the first scatter 308 and an amount of the second scatter 314, in various cases, can be represented by a metric indicative of an amount of the incident light 304 that is transmitted through at least a portion of the brain 312. In various cases, the metric is related to an amount of oxygenated blood in the brain 312.

Further, a portion of the incident light 304 is transmitted through the superficial layers 306 and the brain 312 and is scattered by one or more ventricles 318 of the subject. For instance, third scatter 320 is scattered, reflected, or otherwise emitted by the ventricles 318, transmitted through the brain 312 and the superficial layers 306, and is received by a third detector 322.

In various implementations, a ventricular state of the subject can be determined based on the second scatter 314 and the third scatter 320. For instance, a discrepancy between an amount of the second scatter 314 detected by the second detector 316 and an amount of the third scatter 320 detected by the third detector 322 relates to an amount of blood in the ventricle(s) 318.

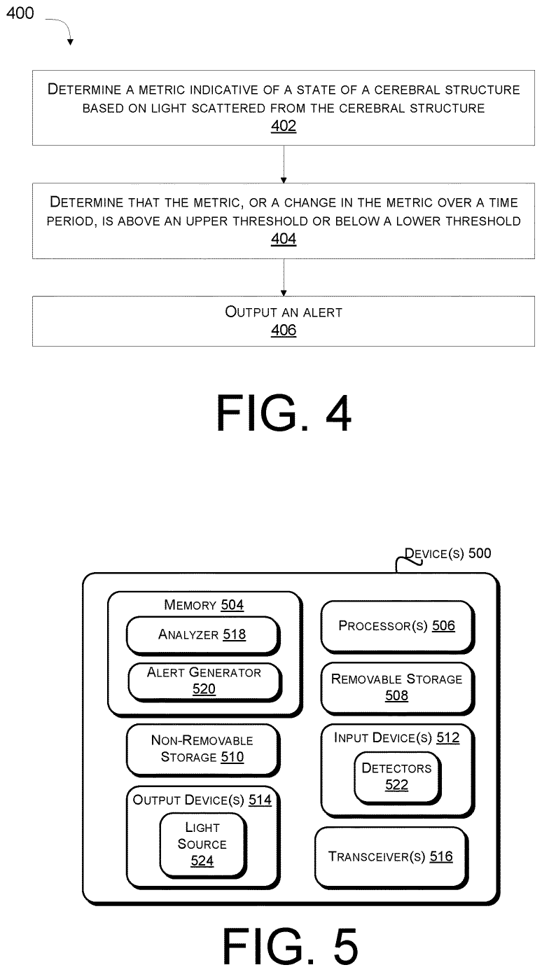

FIG. 4 illustrates an example process 400 for monitoring the state of a cerebral structure. In various cases, the process 400 is performed by an entity, such as the spectroscopy device 104, the monitor 122, at least one processor, one or more computing devices, or any combination thereof.

At 402, the entity determines a metric indicative of a state of a cerebral structure based on light scattered from the cerebral structure. In some examples, the metric is indicative of cerebral oxygenation of a subject. In some cases, the metric is indicative of a ventricular state of the subject. The metric, for example, may be generated based on an amount of light that is transmitted into the head of the subject by a light source and detected by multiple detectors disposed on the surface of the subject. The light source and detectors, in various cases, may be integrated into a spectroscopy device. The spectroscopy device may have an adjustable headband, such that distances between the light source and detectors may be adjustable. In various cases, the spectroscopy device includes at least three detectors.

At 404, the entity determines that the metric, or a change in the metric over a time period, is above an upper threshold or below a lower threshold. In some implementations, if a metric associated with cerebral oxygenation is below a particular threshold, it may be indicative of cerebral hypoxia. In some cases, if a metric associated with an amount of light transmitted through the ventricles of the subject is above a threshold, it may be indicative of the presence of oxygenated blood in the ventricles. In some examples, if a change in the metric during a particular time period is above a particular threshold, then it may be indicative of the presence of blood entering the ventricles.

At 406, the entity outputs an alert. In some implementations, the alert signals, to a care provider, to perform a follow-up test on the subject or to otherwise attend to the subject. In some cases, the alert is an audible alert, a visual alert (e.g., displayed on a screen or other type of display), a haptic alert (e.g., vibration of a haptic feedback device), or any combination thereof.

FIG. 5 illustrates an example of at least one device 500. The device 500 may be part of a spectroscopy device and/or monitor, such as the spectroscopy device 104 and/or monitor 122 illustrated in FIG. 1. The device 500 includes any of memory 504, processor(s) 506, removable storage 508, non-removable storage 510, input device(s) 512, output device(s) 514, and transceiver(s) 516. The device 500 may be configured to perform various methods and functions disclosed herein.

The memory 504 may include one or more components, such as an analyzer 518 and an alert generator 520. The component(s) may include at least one of instruction(s), program(s), database(s), software, operating system(s), etc. In some implementations, the component(s) include instructions that are executed by processor(s) 506 and/or other components of the device 500. For instance, the processor(s) 506, when executing the analyzer 518, may generate one or more metrics described herein based on an amount of light detected by detectors 522. In various cases, the processor(s) 506, when executing the alert generator 520, may generate one or more alerts based on the metric(s).

In some embodiments, the processor(s) 506 include a central processing unit (CPU), a graphics processing unit (GPU), or both CPU and GPU, or other processing unit or component known in the art.

The device 500 may also include additional data storage devices (removable and/or non-removable) such as, for example, magnetic disks, optical disks, or tape. Such additional storage is illustrated in FIG. 5 by removable storage 508 and non-removable storage 510. Tangible computer-readable media can include volatile and non-volatile, removable and non-removable media implemented in any method or technology for storage of information, such as computer readable instructions, data structures, program modules, or other data. The memory 504, the removable storage 508, and the non-removable storage 510 are all examples of computer-readable storage media. Computer-readable storage media include, but are not limited to, Random Access Memory (RAM), Read-Only Memory (ROM), Electrically Erasable Programmable Read-Only Memory (EEPROM), flash memory, or other memory technology, Compact Disk Read-Only Memory (CD-ROM), Digital Versatile Discs (DVDs), Content-Addressable Memory (CAM), or other optical storage, magnetic cassettes, magnetic tape, magnetic disk storage or other magnetic storage devices, or any other medium which can be used to store the desired information and which can be accessed by the device 500. Any such tangible computer-readable media can be part of the device 500.

The device 500 may be configured to communicate over a telecommunications network using any common wireless and/or wired network access technology. Moreover, the device 500 may be configured to run any compatible device Operating System (OS), including but not limited to, Microsoft Windows Mobile, Google Android, Apple IOS, Linux Mobile, as well as any other common mobile device OS.

The device 500 also can include input device(s) 512, such as a keypad, a cursor control, a touch-sensitive display, voice input device, etc. In some cases, the input device(s) 512 include detectors 522 that are configured to detect light. The device 500 may also include output device(s) 514 such as a display, speakers, printers, etc. In some examples, the output device(s) 514 include a light source 524 (e.g., a light source configured to output NIR light or VNIR light).

As illustrated in FIG. 5, the device 500 also includes one or more wired or wireless transceiver(s) 516. For example, the transceiver(s) 516 can include a network interface card (NIC), a network adapter, a Local Area Network (LAN) adapter, or a physical, virtual, or logical address to connect to various network components, for example. To increase throughput when exchanging wireless data, the transceiver(s) 516 can utilize multiple-input/multiple-output (MIMO) technology. The transceiver(s) 516 can comprise any sort of wireless transceivers capable of engaging in wireless, radio frequency (RF) communication. The transceiver(s) 516 can also include other wireless modems, such as a modem for engaging in Wi-Fi, WiMAX, Bluetooth, infrared communication, and the like. The transceiver(s) 516 may include transmitter(s), receiver(s), or both.

### EXPERIMENTAL EXAMPLE

Cerebral near-infrared spectroscopy is a non-invasive tool used to measure regional cerebral tissue oxygenation (rScO₂) initially validated in adult and pediatric populations. Preterm neonates, vulnerable to neurologic injury, are attractive candidates for NIRS monitoring; however, normative data and the brain regions measured by the current technology have not yet been established for this population. This study's aim was to analyze continuous rScO₂ readings within the first 6-72 h after birth in 60 neonates without intracerebral hemorrhage born at ≤1250 g and/or ≤30 weeks' gestational age (GA) to better understand the role of head circumference (HC) and brain regions measured. Using a standardized brain MRI atlas, it was determined that rScO2 in infants with smaller HCs likely measures the ventricular spaces. GA is linearly correlated, and HC is non-linearly correlated, with rScO₂ readings. For HC, it was inferred that rScO2 is lower in infants with smaller HCs due to measuring the ventricular spaces, with values increasing in the smallest HCs as the deep cerebral structures are reached.

### Introduction

Cerebral near-infrared spectroscopy (NIRS) is a widely used monitoring technology that offers the ability to monitor regional cerebral tissue oxygenation (rScO₂), which is postulated to be an expression of cerebral oxygenation and cerebral blood flow (CBF) (Alderliesten, et al., Pediatr. Res. 79, pp. 55-64, 2016). A goal of cerebral NIRS technology is to offer non-invasive continuous monitoring of cerebral tissue oxygenation and extraction, providing clinicians with the prospect of real-time monitoring of the patient's physiological state (Vaidya, R. et al., J. Perinatol. 42, pp. 378-384, 2022; Tataranno, M. L., et al., PLOS One 10, e0124623, 2015; Vesoulis, Z. A., et al., J. Perinatol. 41, pp. 675-688, 2021; Pichler, G., et al., BMJ 380, e072313, 2023). Currently, cerebral NIRS monitoring is employed in many neonatal intensive care units (NICU) across the world (Pichler, G., et al., BMJ 380, e072313, 2023; Chock, V. Y., et al., J. Pediatr. 227, pp. 94-100.e101, 2020; Mintzer, J. P., et al., Pediatr. Res. 86, pp. 296-304, 2019). The technology used for cerebral NIRS was created for adult and pediatric populations; however, it is important to recognize that a large discrepancy exists between adult, pediatric, and neonatal head circumferences (HCs) and tissue composition (Gilmore, J. H., et al., Nat. Rev. Neurosci. 19, pp. 123-137, 2018). There exists a paucity of data and studies evaluating differences in HC and tissue composition when examining baseline rScO₂ in relation to gestational age (GA) of the neonate (Hansen, M. L., et al., Trials 20, pp. 811, 2019). This relationship is particularly important when monitoring preterm infants. Although some normal ranges for rScO₂ during the first 72 h after birth in preterm infants at different GAs have been published, data on normative values remain limited (Alderliesten et al., Pediatr. Res. 79, pp. 55-64, 2016; Pichler, G., et al., J. Pediatr. 163, pp. 1558-1563, 2013; Mohamed, et al., J. Perinatol. 41, pp. 836-842, 2021). It is, however, generally accepted that changes in an individual's own baseline rScO₂ values are associated with alterations in oxygenation and oxygen extraction (Chock, V. Y., et al., J. Pediatr. 227, pp. 94-100.e101, 2020; Mohamed, et al., J. Perinatol. 41, pp. 836-842, 2021; Hyttel-Sorensen, et al., Cochrane Database Syst. Rev. 9, Cd011506, 2017; Hyttel-Sorensen, et al., Trials 14, pp. 120, 2013). The specific brain region(s) measured with existing NIRS technology have not been determined in preterm infants (Vesoulis, Z. A., et al., J. Perinatol. 41, pp. 675-688, 2021). In previous devices utilized for non-infant populations, the NIR beam is assumed to follow a banana-shaped path and measurement depth is thought to be approximately at 50-67% of the distance between emitter and detector (Alderliesten et al., Pediatr. Res. 79, pp. 55-64, 2016; Vaidya, R. et al., J. Perinatol. 42, pp. 378-384, 2022; Tataranno, M. L., et al., PLOS One 10, e0124623, 2015; Vesoulis, Z. A., et al., J. Perinatol. 41, pp. 675-688, 2021).

Currently available neonatal cerebral NIRS sensors measure at an assumed depth of approximately 1.5-2.5 cm, depending on the manufacturer and the type of sensor used (Medtronic, Quick Reference Guide For Premature Neonates, Product Guide, Boulder, CO., 2021; Garvey, A. A., et al., Curr. Opin. Pediatr. 30, pp. 209-215, 2018; Dix, L. M., et al., Pediatr. Res. 74, pp. 557-563, 2013). The actual path length of the NIR beam, which scales with the depth of tissue sampled, remains unknown (Kamran, M. A., et al., Front Neuroinform 12, pp. 37, 2018). Neonatal probes are typically smaller and have a shorter emitter-detector distance compared to the adult sensors (Mintzer, J. P., et al., Pediatr. Res. 86, pp. 296-304, 2019). Since the distance is a fixed parameter, the underlying, corresponding brain region in neonates may differ simply due to their smaller HCs and curvatures. Lack of reliability between rScO₂ readings has been reported when comparing sensors with different emitter-detector distances and also with simple repositioning of the same sensor on the same neonate highlighting the difference and optical heterogeneity of the underlying tissue (Andresen, B., et al., Pediatr. Res. 87, pp. 1244-1250, 2020). Furthermore, tissue maturation, water composition, protein content, lipid content, and vascularization all influence optic properties, and therefore, rScO2 measurements are subject to rapid changes in the still developing neonatal brain compared to an adult (Zhang, Y., et al., Neuroimage 185, pp. 349-360, 2019).

The aim of this experimental example is to evaluate continuous rScO2 readings within the first 72 h after birth in preterm infants without overt neurologic injury and to determine whether these readings were altered by GA, HC, or different brain regions captured by the commercially available neonatal sensor of the INVOS™ 5100C system.

### Methods

### Patient Selection

This experimental example was an observational single-center cohort study of preterm infants born at ≤1250 g and/or ≤30 weeks' gestation and admitted following delivery to the University of Washington Medical Center NICU between May 2019 and May 2021. Exclusion criteria were <66 h of continuous rScO2 monitoring, major congenital anomalies, death within 72 h after birth, HC>30 cm (Z-score>3), and intracranial hemorrhage (ICH) diagnosed by cranial ultrasound 7-10 days after birth as half of these hemorrhages are likely to have occurred by the first day of life.20 The study was approved by the Institutional Review Board at the University of Washington School of Medicine UW IRB #00006091.

### Data Collection

Demographic and clinical data collection occurred retrospectively from the medical record and included administration of prenatal corticosteroids, maternal pre-eclampsia, maternal chorioamnionitis, birth weight, GA, HC obtained within 1 h of birth (birth HC), next recorded HC after initial birth HC, small for GA defined as birth weight<10th percentile, Apgar score at 1 and 5 min after birth, respiratory support type at birth, first diastolic blood pressure after birth, first hematocrit, vasopressor use, and lowest and highest measured pCO₂ in the first 72 h after birth. For the first hematocrit, the value from the day of birth was used; if no value was available on day of birth, the value from day 1 after birth was used. For the first documented diastolic blood pressure, an arterial measurement was used; if no arterial measurement was available, a non-invasive measurement was utilized. Diastolic blood pressure was examined as CBF in preterm neonates has been shown to be highly reliant on diastolic blood flow.

### NIRS Monitoring and Technology

Infants were monitored prospectively with continuous cerebral NIRS using the INVOS™ 5100C regional oximeter with INVOS cerebral oximetry infant-neonatal sensor (Medtronic, Minneapolis) during the first 72 h after birth. The infant-neonatal sensor has two detectors: a shallow detector spaced 3 cm from the emitter and a deep detector spaced 4 cm from the emitter. The shallow detector measures at a depth of approximately 1.5 cm, whereas the deep detector measures at a depth of approximately 2.5 cm. The measurement depth is derived using spatially resolved spectroscopy, subtracting shallow detector measurement from the deep detector measurement to remove the interference of measurements from superficial structures such as the skin, muscle, bone, and axial spaces, providing a reading at approximately 2.5 cm depth (Medtronic, Quick Reference Guide For Premature Neonates, Product Guide, Boulder, CO., 2021; Covidien, Operations Manual INVOS® System, Model 5100C, 2013). The infant-neonatal sensor was placed on the neonate's forehead within the first 6 h after birth. It was chosen to examine data from 6 h onward to best harmonize data analysis across the entire cohort. rScO2 data were captured in 5-10-s intervals and exported via INVOS™ Analytics Tool version 1.2.1 (Medtronic, Minneapolis).

### Measurements of Fetal MRI

Fetal T2 MRI brain images were provided by the Harvard Fetal Brain Atlas (http://crl.med.harvard.edu/research/fetal_brain_atlas/), accessed Mar. 15, 2022. Each image pixel represents an area of 0.8 mm², allowing for an elliptical arch to be drawn corresponding with the infrared ray detector and arch depth measurements listed above. The depths of the rays were adjusted to account for scalp and skull depth, which in neonates and newborns has been measured as less than 1 mm (Li, Z., et al., PLOS One 10, e0127322, 2015). As the path of the ray is influenced by multiple factors including myelination and water content of the structures through which the ray is passing, these arches are provided as estimates. Image analysis and measurements were performed using FMRIB Software Library (FSL v6.0, Oxford, UK, https://fsl.fmrib.ox.ac.uk/fsl/fslwiki/FslInstallation), accessed Nov. 23, 2021 (Gholipour, A., et al., Sci. Rep. 7, pp. 476, 2017; Jenkinson, M., et al., Neuroimage 62, pp. 782-790, 2012; Computational Radiology Laboratory at Boston Children's Hospital. (ed. Gholipour, A.), Harvard Fetal Brain Atlas (Boston, MA), 2017). Graphical illustration and coloring of the arches was performed using Microsoft PowerPoint (2019, Version 2211 Build 16.0.15831.20098).

### Statistical Analysis

Descriptive statistics were used to describe the demographic variables of the included infants using either median and interquartile range (IQR) or count and percentage. rScO₂ data were averaged into hourly epochs by determining the median value for each infant in each epoch. To visually examine how rScO₂ changes with HC and GA, continuous rScO₂ curves were generated using locally estimated scatterplot “loess” fitting with either standard deviation (aggregated trajectories) or 95% confidence intervals (CI, individual patient trajectories) starting 6 h after birth. Trajectories of rScO₂ were plotted for three HC groups (18-21.9, 22-25.9, and ≥26 cm) and four GA groups (22-24, 25-27, 27-29, and ≥30 weeks). For GA plots, infants with an HC Z-score more than ±2 were excluded (n=7). To examine the population effects of GA and HC on rScO2 over time, a generalized estimating equations (GEE) approach was used (Liang, K. Y., et al., Annu. Rev. Public Health 14, pp. 43-68, 1993). Linear GEE models accounting for HC, GA, hematocrit, and time (as hourly epoch) were constructed, with clustering by infant and an exchangeable correlation structure. Both HC was modeled a third-order polynomial, with time epoch as a second-order polynomial and hematocrit and GA as linear continuous variables. A multivariate Wald test was used to compare models with and without the HC terms. Daily hematocrit was used and aligned with the respective epochs, with missing values replaced with the nearest available hematocrit level. P values<0.05 were considered statistically significant. Statistical analyses were performed in R version 4.1.2 in the R studio environment (R: A Language and Environment for Statistical Computing (R Core Team), 2019).

### Results

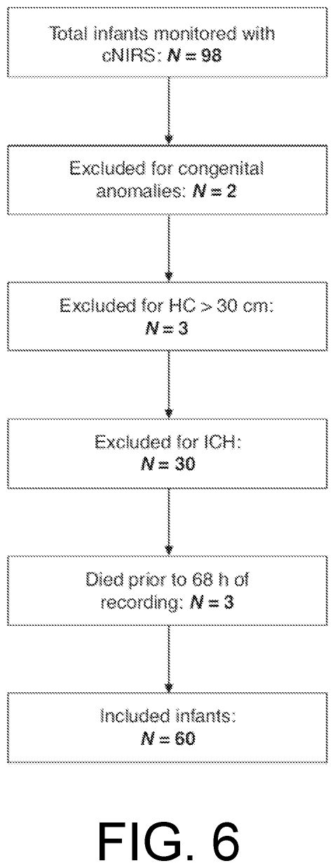

During the study period, 98 infants were monitored with cerebral NIRS. Of those, 2 infants (2.0%) with congenital abnormalities were excluded, 3 infants (3.1%) with HCs>30 cm were excluded, 4 infants (4.1%) who died prior to 72 h of NIRS monitoring were excluded, and 30 infants (30.6%) who were diagnosed with ICH were excluded. The remaining 60 (61.2%) infants were included in the analysis (FIG. 6). Baseline characteristics of the study population are depicted in Table 1. Median (IQR) number of hours of data collection in the 60 infants was 66 (65.5-66), indicating that most infants had data for the entire 6-72 h period after birth. No significant difference between HC at birth (median 24.5 cm, IQR 23.4-26.5) and next measured HC (median 24.0 cm, IQR 23.0-25.95) were found.

### Determination of Estimated NIRS Sensor Measurement by Head Circumference and Gestational Age

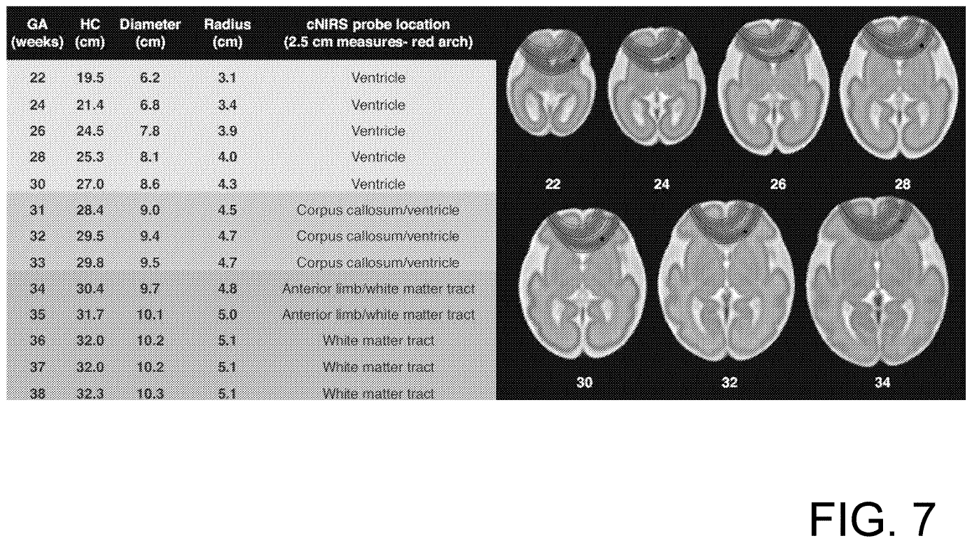

First, the anatomical structures located at a signal depth of 2.5 cm were determined using a fetal MRI atlas as a reference guide for normal brain development. The anatomical structures captured 2.5 cm from the forehead, where the NIRS probe was placed, are listed in the table in FIG. 7. Representative images in FIG. 7 depict the approximate trajectory of where the rScO₂ reading arises for a given HC-the starred arc estimates the light path for the deep sensor and non-starred arc estimates the light path for the shallow sensor. In infants with HC<30 cm, there is a high probability that at least a portion of the rScO₂ readings reflects data from ventricular cerebrospinal fluid (CSF). For infants with an HC<27 cm, the majority of the rScO₂ reading with the device tested is likely to come from the ventricle and deep brain structures.

Based on the results in FIG. 7, infants were grouped by the anatomical structures estimated to be measured most frequently within a given HC and GA. This resulted in three groups for rScO₂ trajectory analysis as follows—Group 1:18-21.9 cm with an estimated measurement level at the deep gray matter structures, Group 2:22-25.9 cm with the estimated measurement of the cerebral NIRS sensor coming from the ventricle, and Group 3:26-30 cm, with an estimated measurement region of cortex and white matter.

rScO2 in Relation to Head Circumference and Gestational Age

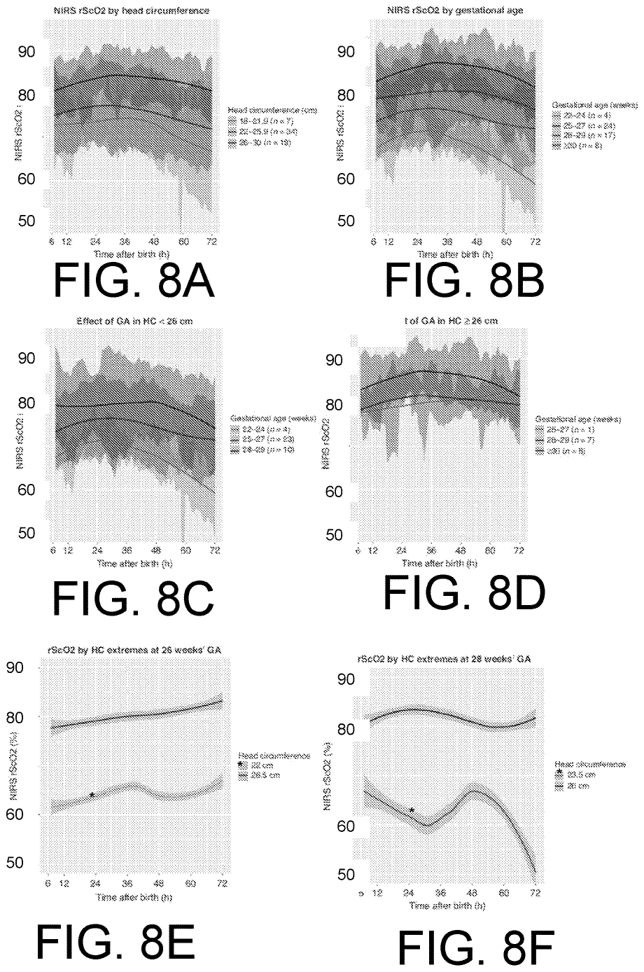

The median rScO₂ trajectory over the first 6-72 h after birth varied significantly based on different HCs. Infants with the largest HC had the highest rScO₂ readings and average rScO₂ tended to decrease with decreasing HC (FIG. 8A). Similar trajectories of rScO₂ were observed in relation to GA. The most immature infants had the lowest measured rScO₂ trajectories, whereas the more mature infants had higher rScO2 measurements (FIG. 8B). While the relationship between GA and rScO₂ was linear, HC was non-linearly associated with rScO2. In particular, rScO₂ appeared to decrease with decreasing HC, but flattened or potentially began to increase again as HC decreased below 22 cm.

Influence of Gestational Age on rScO2 in Infants With Similar Head Sizes

To examine the role of changes in tissue composition with increasing GA on rScO₂ measurement, the neonates were grouped into two categories: Group 1 HC<26 cm, and Group 2 HC≥26 cm. The two categories were derived by determining that those with HC≥26 cm likely had rScO2 readings in part in the cortex (FIG. 7). As expected, higher GA was associated with higher rScO₂ measurements even after accounting for HC grouping (FIG. 8C, FIG. 8D); however, the range of rScO₂ measurements in neonates with HC<26 cm was 59-85% across all GAs compared to 76-87% in neonates with HC>26 cm, demonstrating a dominant effect of HC on rScO₂ measurement (FIG. 8C, FIG. 8D). Similarly with matched GA infants at 26 and 28 weeks, those with larger HC had higher rScO2 measurements (FIG. 8E, FIG. 8F).

After Accounting for GA, HC Also Contributes to rScO2 Readings

In the fully adjusted GEE model, terms for HC, GA, and hematocrit were all significantly associated with the median rScO₂ value. Using a multivariate Wald test, the HC terms were statistically significant (p=0.031), suggesting that HC is an important contributor to rScO2 after accounting for complex relationships with GA and time, as well as adjusting for hematocrit.

### Discussion

As the field of neonatology continues to advance and the lower limit of viable GA continues to decrease, clinicians are faced with technologies that are not completely adapted or validated to their youngest patients. In the case of cerebral NIRS, this includes smaller head sizes which results in considerable variation in the brain region being measured by current cerebral NIRS sensors. As rScO2 readings are increasingly incorporated into clinical decision making, it can be important to understand the brain region captured in preterm infants. In this study, rScO2 trajectories over the first 6-72 h after birth are presented in a cohort of 60 preterm infants born at ≤1250 g or <30 weeks gestation without ICH. It is shown that in these first 6-72 h, the expected rScO₂ trajectory is different when stratified by HC independent of GA, emphasizing the importance of the tissue being measured. In particular, rScO₂ readings by the device tested in infants with HCs<26 cm are likely to have a higher proportion of the rScO₂ measurement by ventricular content, therefore not consistently reflecting cerebral tissue oxygenation itself (FIG. 7). In the smallest heads, rScO₂ by the device tested may then incorporate greater proportions of deep gray matter, with independent non-linear effects of HC on rScO₂ after taking into account GA and hematocrit.

In a cerebral NIRS probe, light is detected by proximal and distal detectors, which allows for the processing of the shallow and deep optical signals separately to provide a spatial resolution; data from the scalp and surface tissue are subtracted and suppressed to provide a reading that reflects rScO₂ in deeper tissues (Covidien, Operations Manual INVOS® System, Model 5100C, 2013; Duncan, A., et al., Phys. Med. Biol. 40, pp. 295-304, 1995; Demel, A., et al., J. Biomed. Opt. 19, pp. 17004, 2014; Ostojic, D., et al., Adv. Exp. Med. Biol. 1232, pp. 33-38, 2020; Delpy, D. T., et al., Phys. Med. Biol. 33, pp. 1433-1442, 1988). Unlike the adult, pediatric, or even term newborn, the region of rScO₂ reading in preterm infants is unlikely to be the superficial cortex and may frequently extend through the ventricles into the deep gray matter (FIG. 7). For example, the average HC of term neonates is 35 cm with a radius of 5.5 cm (assuming a perfectly round head shape), and an average adult HC is 55 cm with a radius of 8.7 cm (Jaekel, J., et al., J. Int. Neuropsychol. Soc. 25, pp. 48-56, 2019; Bushby, K. M., et al., Arch. Dis. Child 67, pp. 1286-1287, 1992). In the experimental cohort, the smallest HC was 18 cm, which equates to a radius of 2.9 cm. Therefore, rScO₂ measurements reflect deeper cerebral structures rather than just the cortical white matter. Given that light absorption through the skull and extracerebral tissues is negligible in neonates, the NIR beam projects into the ventricles and deep gray matter (Demel, A., et al., J. Biomed. Opt. 19, pp. 17004, 2014; Ostojic, D., et al., Adv. Exp. Med. Biol. 1232, pp. 33-38, 2020). The different anatomical structures of the brain also vary in composition. As an extreme example, CSF in the ventricles clearly has a different composition and oxygen content relative to white matter structures measured in larger HCs or deep gray matter structures measured in smaller HCs (Zhang, Y., et al., Neuroimage 185, pp. 349-360, 2019; Delpy, D. T., et al., Phys. Med. Biol. 33, pp. 1433-1442, 1988; Li, T., et al., J. Biomed. Opt. 16, pp. 045001, 2011). Although NIRS technology was created to measure regional tissue oxygen bound to hemoglobin, CSF itself has been shown to alter the propagation of light in NIRS models (Delpy, D. T., et al., Phys. Med. Biol. 33, pp. 1433-1442, 1988; Li, T., et al., J. Biomed. Opt. 16, pp. 045001, 2011; Hoshi, Y., J. Biomed. Opt. 12, pp. 062106, 2007; Okada, E., et al., Appl Opt. 36, pp. 21-31, 1997; Okada, E., et al., Appl Opt. 42, pp. 2906-2914, 2003). These results support the potential for CSF (or the absence of other tissue) to impact the rScO₂ reading, with a greater proportion of CSF included at the 2.5 cm measurement depth in infants with smaller HCs (FIG. 7), which is associated with lower rScO2 independently of GA (FIGS. 8A to 8F).

Despite the same wavelength, optical path length differs in adult and neonatal heads (average of 6.26 vs 4.99 cm, respectively) and can vary in the same subject with changes in tissue geometry, tissue water content, and hemoglobin concentration (Duncan, A., et al., Phys. Med. Biol. 40, pp. 295-304, 1995; Owen-Reece, H., et al., Br. J. Anaesth. 82, pp. 418-426, 1999; Yoshitani, K., et al., Anesth. Analg. 104, pp. 341-346, 2007). Tissue composition and myelin content change throughout neonatal developmental stages and also affect the brain's optical properties. For example, the penetration depth is highly dependent on the scattering effect of myelin which is not well developed in the extreme preterm brain; therefore, penetration depth of the emitting light might be even deeper than the expected 2.5 cm (Zhang, Y., et al., Neuroimage 185, pp. 349-360, 2019; Svaasand, L. O., et al., Photochem. Photobio. 38, pp. 293-299, 1983). This process is dynamic, with myelination starting in the brainstem around 20 weeks of gestation and progressing rapidly alongside a concurrent decrease in ventricle size (Zhang, Y., et al., Neuroimage 185, pp. 349-360, 2019; Barkovich, A. J., J. Inherit. Metab. Dis. 28, pp. 311-343, 2005). Furthermore, deep gray matter has differences in optical properties compared to white matter, which may account for further variations in rScO₂ based on the region of measurement (Li, T., et al., J. Biomed. Opt. 16, pp. 045001, 2011). Thus, not only is head size and physical location of the cerebral NIRS probe relevant when interpreting values, but the change of maturational tissue composition may also be relevant when evaluating rScO₂ measurements over time.

The anatomical structures captured also vary in their vasculature and microvasculature, which results in a difference of the mixed arterial and venous signal for which NIRS is engineered to report (Suppan, E., et al., Front. Pediatr. 10, pp. 913223, 2022). rScO₂ is calculated from the difference between arterial and venous oxygenation (fSO₂=0.25×SaO2+0.75×SvO₂), which changes with age (Suppan, E., et al., Front. Pediatr. 10, pp. 913223, 2022). While this 25:75 constant of arterial to venous blood is applicable to adults, pediatric patients have a constant of 15:85, and no normative values have been established in (preterm) neonates (Suppan, E., et al., Front. Pediatr. 10, pp. 913223, 2022). Furthermore, regional trends in rScO 2 at lower GAs differ from those of the peripheral vasculature, perhaps due to variations in the arterial: venous constant in cerebral tissue (Suppan, E., et al., Front. Pediatr. 10, pp. 913223, 2022). The relatively larger proportion of venous volume at younger GAs may partially explain why cerebral rScO2 values are lower in these infants and may be further exacerbated by differences in developmental stages of vascularization in the brain region measured (Kratzer, I., et al., Front. Neurosci. 8, pp. 359, 2014). For example, a recent study using pulse sequence MRI in neonates found that veins draining the central brain had 5% lower cerebral venous oxygenation (65% compared to 70%; p=0.02) than veins draining cortical brain (Jiang, D., et al., Magn. Reson. Med. 82, pp. 1129-1139, 2019). This may provide an additional mechanistic explanation of how venous differences could help account for lower rScO2 measurements in neonates with smaller HCs in whom the probe measures more centrally (FIG. 8B).

Additionally, fluctuations in cerebral arterial and venous volume in neonates are unique during the transitional period immediately following birth (Suppan, E., et al., Front. Pediatr. 10, pp. 913223, 2022; Schwaberger, B., et al., Front. Pediatr. 6, pp. 132, 2018). As depicted in FIG. 8, rScO₂ measurements increase around 12-14 h and peak at 24-30 h. These transitions in rScO₂ differ clinically from the peripheral venous saturation, which transitions and stabilizes within minutes after birth (Dawson, J. A., et al., Pediatrics 125, e1340-e1347, 2010). Changes in rScO₂ over time may result from a known transient decrease in arterial CBF after birth that then increases again by 24 h after birth (Baytur, Y. B., et al., Ultrasound Obstet. Gynecol. 24, pp. 522-528, 2004). In addition, if the arterial component is less than the empirically derived 25% by the INVOS technology, the decrease in the reading might be artifactual (Medtronic, Quick Reference Guide For Premature Neonates, Product Guide, Boulder, CO., 2021; Covidien, Operations Manual INVOS® System, Model 5100C, 2013). This alteration appears to be more pronounced at lower GAs, which is consistent with the inverse relationship between CBF autoregulation and GA (Rhee, C. J., et al., Pediatr. Res. 84, pp. 602-610, 2018; Schwaberger, B., et al., Front. Pediatr. 6, pp. 132, 2018; Baytur, Y. B., et al., Ultrasound Obstet. Gynecol. 24, pp. 522-528, 2004; Baik, N., et al., Neonatology 112, pp. 97-102, 2017). The rScO₂ unique shape during the transitional period may also be accounted for by a GA-related decrease in vascular resistance and subsequent increase in venous circulation following delivery (FIGS. 8A to 8F) (Laurichesse-Delmas, H., et al., Ultrasound Obstet. Gynecol. 13, pp. 34-42, 1999).

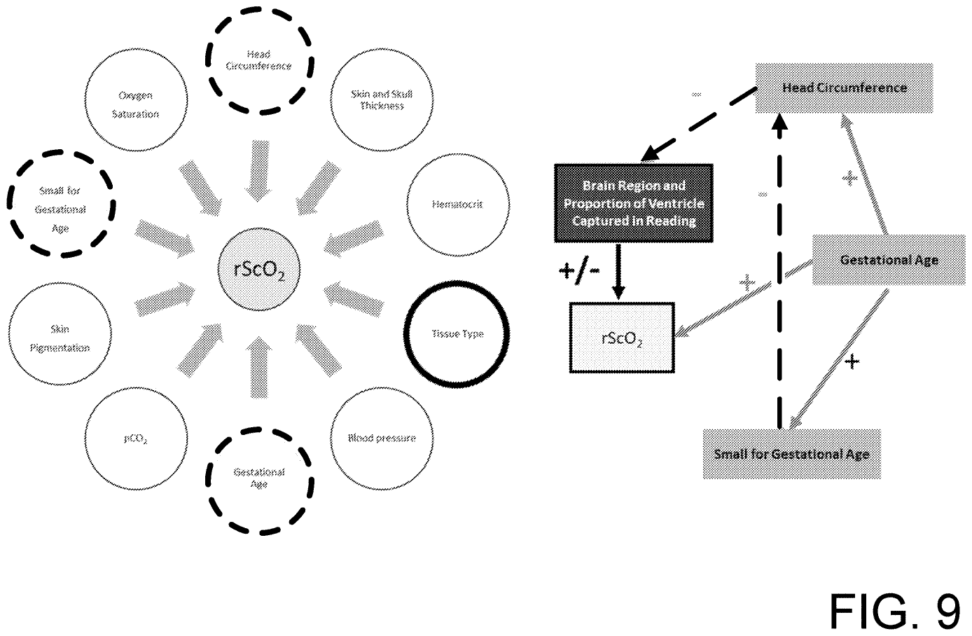

NIRS technology relies on light absorption by hemoglobin, using near-infrared light at wavelengths 730 and 810 nm to allow absorption by hemoglobin (Covidien, Operations Manual INVOS® System, Model 5100C, 2013). Furthermore, a higher hematocrit, has been positively correlated to rScO2 readings in pediatric and neonatal patients (Sood, B. G., et al., Semin. Fetal Neonatal Med. 20, pp. 164-172, 2015; Baenziger, O., et al., Pediatrics 119, pp. 455-459, 2007; Tobias, J. D., J. Clin. Monit. Comput. 25, pp. 171-174, 2011). In this study, hematocrit was positively correlated and thus accounted for when deciphering the HC and rScO2 association. Alderliesten et al. did not find an association between HC and rScO₂ and thus postulated that differences in CBF account for the observed differences between infants of different GAs; however, their data did not account for the potential contribution of differing tissue type and composition nor did their study differentiate between infants with and without ICH (Alderliesten et al., Pediatr. Res. 79, pp. 55-64, 2016). Since NIRS probes in preterm infants may be capturing an area filled with CSF, blood within the CSF would be expected to alter rScO2 as suggested by recent evidence (Hyttel-Sorensen, et al., Trials 14, pp. 120, 2013; Laurichesse-Delmas, H., et al., Ultrasound Obstet. Gynecol. 13, pp. 34-42, 1999; Kurth, C. D., et al., Anesth. Analg. 84, pp. 1297-1305, 1997). Therefore, any studies of rScO2 in premature infants must account for influence of intraventricular hemoglobin (Zhang, Y., et al., Annu. Int. Conf. IEEE Eng. Med. Biol. Soc. 2011, pp. 1937-1940, 2011; Pavlek, L. R., et al., Front. Pediatr. 8, pp. 624113, 2020; Beausoleil, T. P., et al., Sci. Rep. 8, pp. 6511, 2018; Korc̆ek, P., et al., J. Perinatol. 37, pp. 1070-1077, 2017). In addition, although NIRS technology is designed to measure the concentration changes of oxy and deoxy-hemoglobin, the presence of hemoglobin has not been shown to be a requirement for getting a reasonable reading—it has been described that NIRS readings are still possible in metabolically inactive tissues from cadavers and objects void of hemoglobin such as vegetables (Svaasand, L. O., et al., Photochem. Photobio. 38, pp. 293-299, 1983; Kahn, R. A., et al., Eur. J. Anaesthesiol. 35, pp. 907-910, 2018). The high prevalence of ICH in preterm infants, and the likelihood that a blood clot resulting from an ICH would be an important confounder when examining rScO₂ from infants with smaller HCs where readings include the ventricles, suggests that any studies of cerebral NIRS in premature infants must account for the impact of ICH (Zhang, Y., et al., Annu. Int. Conf. IEEE Eng. Med. Biol. Soc. 2011, pp. 1937-1940, 2011; Pavlek, L. R., et al., Front. Pediatr. 8, pp. 624113, 2020; Beausoleil, T. P., et al., Sci. Rep. 8, pp. 6511, 2018; Korc̆ek, P., et al., J. Perinatol. 37, pp. 1070-1077, 2017). FIG. 9 illustrates secondary characteristics that have been shown to influence rScO₂ measurements.

In summary, this experimental example objectively describes how HC and rScO₂ readings are positively but non-linearly correlated in preterm infants, which may be due to variation in the brain region the cerebral NIRS probe is sensing at a fixed distance. In addition, rScO₂ trajectories in preterm infants may be dependent on unique cerebral physiologies of this population. Both GA and HC are relevant in determining cerebral oxygenation in this population. In various cases, a spectroscopy device utilizes one or more of the secondary characteristics illustrated in FIG. 9 to enhance the accuracy of spectroscopy-based measurements (e.g., cerebral oxygenation).

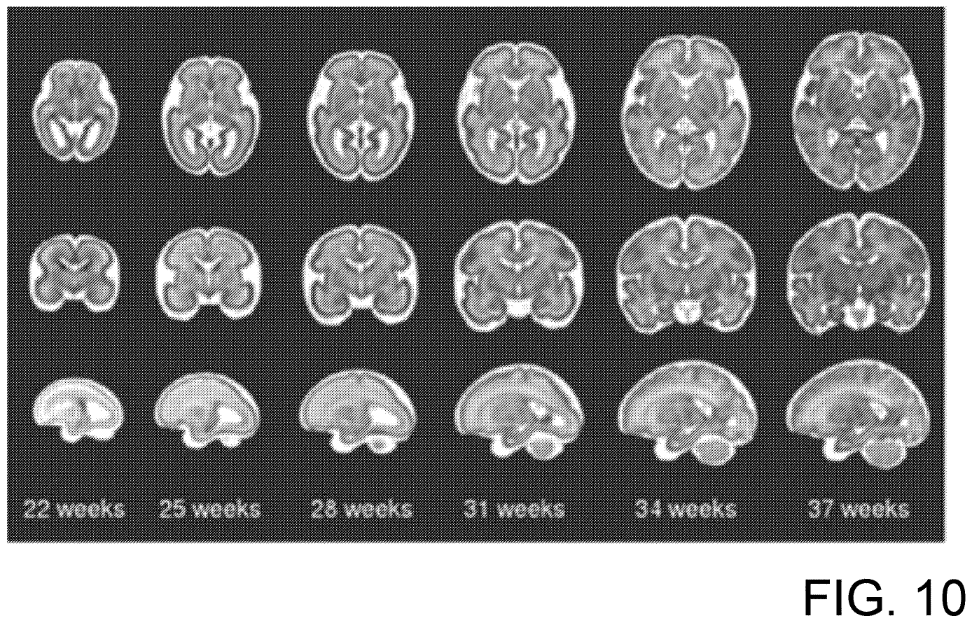

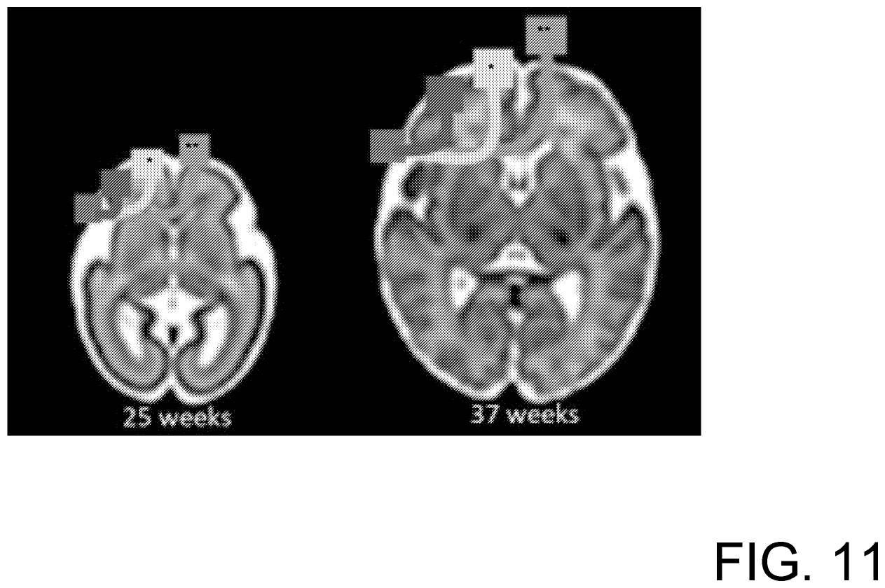

In various implementations, a spectroscopy device with adjustable distances between light source and detectors may enable accurate readings for preterm infants. FIG. 10 illustrates the different cerebral physiology of infants at different gestational ages. Because the different cerebral physiology may result in different NIR propagation patterns and absorbance, gestational age can be a factor to accurately determine cerebral oxygenation and ventricular state of subjects. FIG. 11 illustrates an example of the propagation patterns for a spectroscopy device that is suitable for infants at 25- and 37-week gestational ages. For instance, to implement a propagation path that travels through the ventricles of both 25- and 37-week infants, the distances between light emitter and light detectors should be adjustable. Furthermore, the relatively significant penetration depth of NIR light for this population may enable ventricular monitoring using spectroscopy.

### Example Clauses

1. A cerebral spectroscopy monitoring device, including: an adjustable headband configured to be disposed on an external surface of a head of a neonatal subject; a light source physically coupled to the adjustable headband and configured to output visible and near infrared (VNIR) light through a skull of the neonatal subject; a first detector physically coupled to the adjustable headband and configured to detect a first portion of the VNIR light scattered from and/or propagated through at least one of skin, muscle, or the skull of the neonatal subject; a second detector physically coupled to the adjustable headband and configured to detect a second portion of the VNIR light scattered from and/or propagated through a brain tissue of the neonatal subject; a third detector physically coupled to the adjustable headband and configured to detect a third portion of the VNIR light scattered from and/or propagated through a ventricle of the neonatal subject; at least one output device configured to output an indication of a state of the brain tissue based on the first portion, the second portion, and third portion; and a processor configured to: detect an intraventricular hemorrhage of the neonatal subject by detecting a change in the third portion over time; and in response to detecting the intraventricular hemorrhage, cause the at least one output device to output an alert.
2. The device of clause 1, wherein the neonatal subject is a preterm infant.
3. The device of clause 1 or 2, wherein the neonatal subject is 72 hours old or less.
4. The device of any of clauses 1 to 3, wherein the adjustable headband includes: an adjustable portion disposed between the light source and at least one of the first detector, the second detector, or the third detector.
5. The device of any of clauses 1 to 4, wherein the adjustable headband includes: an adjustable portion disposed between the first detector and the second detector.
6. The device of any of clauses 1 to 5, wherein the adjustable headband includes: an adjustable portion disposed between the second detector and the third detector.
7. The device of any of clauses 1 to 6, wherein the adjustable headband includes an adjustable snap mechanism and/or an elastic material.
8. The device of any of clauses 1 to 7, further including: a biocompatible adhesive disposed on a surface of the adjustable headband, the biocompatible adhesive being configured to adhere the adjustable headband to the skin of the neonatal subject.
9. The device of clause 8, wherein the biocompatible adhesive includes a hydrocolloid.
10. The device of any of clauses 1 to 9, wherein the first portion is emitted from a tissue that is disposed below the surface of the skull of the neonatal subject by a depth of about 1 cm to about 2 cm.
11. The device of any of clauses 1 to 10, wherein the second portion is emitted from a tissue that is disposed below the surface of the skull of the neonatal subject by a depth of about 1 cm to about 8 cm.
12. The device of any of clauses 1 to 11, wherein the third portion is emitted from a structure that is disposed below the surface of the skull of the neonatal subject by a depth of about 1.5 cm to about 10 cm.
13. The device of any of clauses 1 to 12, wherein a distance between the light source and the first detector along the adjustable headband is in a range of about 1 cm to about 5 cm.
14. The device of any of clauses 1 to 13, wherein a distance between the light source and the second detector along the adjustable headband is in a range of about 1.5 cm to about 7.5 cm.
15. The device of any of clauses 1 to 14, wherein a distance between the light source and the third detector along the adjustable headband is in a range of about 2 to about 10 cm.
16. The device of any of clauses 1 to 15, wherein a distance between the first detector and the second detector along the adjustable headband is in a range of about 0.5 cm to about 2.5 cm.
17. The device of any of clauses 1 to 16, wherein a distance between the second detector and the third detector along the adjustable headband is in a range of about 0.5 to about 2.5 cm.
18. The device of any of clauses 1 to 17, wherein the indication of the state of the brain tissue includes a regional blood oxygenation of blood traversing the brain tissue, and wherein the processor is further configured to: determine the regional blood oxygenation based on the second portion; and correct the regional blood oxygenation based on the first portion and the third portion.
19. The device of clause 18, wherein the processor is configured to determine the regional blood oxygenation of the blood traversing the brain tissue further based on at least one of: an age of the neonatal subject; a weight of the neonatal subject; a head circumference of the neonatal subject; a skin pigmentation of the neonatal subject; a bilirubin state of the neonatal subject; an oxygen saturation of the neonatal subject; a partial pressure of carbon dioxide (pCO₂) in blood of the neonatal subject; a blood pressure of the neonatal subject; a skin thickness of the neonatal subject; a skull thickness of the neonatal subject; or a hematocrit of the neonatal subject.
20. The device of clause 19, wherein the processor is further configured to: determine that the regional blood oxygenation is above an upper threshold or below a lower threshold; and in response to determining that the regional blood oxygenation is above the upper threshold or below the lower threshold, generating an alert, and wherein the at least one output device is further configured to output the alert.
21. The device of any of clauses 1 to 20, wherein the processor is further configured to determine a state of the ventricle by subtracting a magnitude of the first portion and a magnitude of the second portion from a magnitude of the third portion.
22. The device of clause 21, wherein the at least one output device is further configured to output an indication of the state of the ventricle.
23. The device of clause 21 or 22, wherein the processor is further configured to: determine that a metric indicative of the state of the ventricle is above an upper threshold or below a lower threshold; and in response to determining that the metric is above the upper threshold or below the lower threshold, generating an alert, and wherein the at least one output device is further configured to output the alert.
24. The device of any of clauses 21 to 23, wherein the processor is further configured to: determine a change in a metric based on at least one of the first portion, the second portion, or the third portion over a time interval; determine that the change in the metric is above an upper threshold or below a lower threshold; and in response to determining that the change in the metric is above the upper threshold or below the lower threshold, generating an alert, wherein the at least one output device is configured to output the alert.
25. The device of any of clauses 1 to 24, wherein the device further includes: a sensor configured to detect a physiological parameter of the neonatal subject, and wherein the at least one output device is further configured to output an indication of the physiological parameter.
26. The device of clause 25, wherein the physiological parameter includes a level of blood glucose, a hematocrit, a pulse, an EEG, a blood pressure, a pulse oxygenation, an airway parameter, an ECG, a blood flow, a pCO₂, or a temperature.
27. A computing device, including: at least one processor; and memory storing instructions that, when executed by the at least one processor, cause the at least one processor to perform operations, including: receiving first data indicative of a first portion of VNIR light detected by a first detector; receiving second data indicative of a second portion of the VNIR light detected by a second detector; receiving third data indicative of a third portion of the VNIR light detected by a third detector; determining a metric indicative of a state of a brain tissue based on the first data, the second data, and the third data; determining a metric indicative of a state of a ventricle based on the first data, the second data, and the third data; and outputting a signal based on the metric indicative of the state of the brain tissue and/or the metric indicative of the state of the ventricle.
28. The computing device of clause 27, wherein the metric indicative of the state of the brain tissue includes a regional oxygenation of the brain tissue.
29. The computing device of clause 27 or 28, wherein determining the metric indicative of the state of the brain tissue is further based on a secondary characteristic including at least one of: an age of a subject; a weight of the subject; a head circumference of the subject; a skin pigmentation of the subject; a bilirubin state of the subject; an oxygen saturation of the subject; a partial pressure of carbon dioxide (pCO₂) in blood of the subject; a blood pressure of the subject; a skin thickness of the subject; a skull thickness of the subject; or a hematocrit of the subject.
30. The computing device of clause 29, further including: at least one input device configured to receive a signal indicative of the secondary characteristic.
31. The computing device of any of clauses 27 to 30, further including: determining that the metric indicative of the state of the brain tissue is above an upper threshold or below a lower threshold; wherein outputting the signal based on the metric indicative of the state of the brain tissue includes, in response to determining that the metric indicative of the state of the brain tissue is above the upper threshold or below the lower threshold, outputting an alert.
32. The computing device of any of clauses 27 to 31, further including: determining that the metric indicative of the state of the ventricle is above an upper threshold or below a lower threshold; wherein outputting the signal based on the metric indicative of the state of the ventricle includes, in response to determining that the metric indicative of the state of the ventricle is above the upper threshold or below the lower threshold, outputting an alert.
33. The computing device of any of clauses 27 to 32, further including: determining that a change in the metric indicative of the state of the brain tissue over a time interval is above an upper threshold or below a lower threshold; wherein outputting the signal based on the metric indicative of the state of the brain tissue includes, in response to determining that the change is above the upper threshold or below the lower threshold, outputting an alert.
34. The computing device of any of clauses 27 to 33, further including: determining that a change in the metric indicative of the state of the ventricle over a time interval is above an upper threshold or below a lower threshold; wherein outputting the signal based on the metric indicative of the state of the ventricle includes, in response to determining that the change is above the upper threshold or below the lower threshold, outputting an alert.
35. The computing device of any of clauses 27 to 34, further including: a display configured to visually present the signal.
36. The computing device of any of clauses 27 to 35, further including: a speaker configured to audibly output the signal.
37. The computing device of any of clauses 27 to 36, further including: a transceiver configured to transmit the signal to an external device.
38. A spectroscopy device, including: an adjustable headband; a light source physically coupled to the adjustable headband and configured to output visible and near infrared (VNIR) light; a first detector physically coupled to the adjustable headband and configured to detect a first portion of the VNIR light; a second detector physically coupled to the adjustable headband and configured to detect a second portion of the VNIR light; and a third detector physically coupled to the adjustable headband and configured to detect a third portion of the VNIR light, the second detector being disposed between the first detector and the third detector along the adjustable headband.
39. The spectroscopy device of clause 38, further including an adhesive disposed on the adjustable headband.
40. The spectroscopy device of clause 38 or 39, wherein the adjustable headband includes an adjustable snap mechanism and/or an elastic material.
41. The spectroscopy device of clause 40, wherein the adjustable snap mechanism and/or the elastic material is disposed between at least one of: the light source and the first detector; the first detector and the second detector; or the second detector and the third detector.
42. The spectroscopy device of any of clauses 38 to 41, wherein a distance between the light source and the first detector along the adjustable headband is adjustable within a range of about 1 cm to about 5 cm.
43. The spectroscopy device of any of clauses 38 to 42, wherein a distance between the light source and the second detector along the adjustable headband is adjustable within a range of about 1.5 cm to about 7.5 cm.
44. The spectroscopy device of any of clauses 38 to 43, wherein a distance between the light source and the third detector along the adjustable headband is adjustable within a range of about 2 cm to about 10 cm.
45. The spectroscopy device of any of clauses 38 to 44, wherein a distance between the first detector and the second detector along the adjustable headband is adjustable within a range of about 0.5 cm to about 2.5 cm.
46. The spectroscopy device of any of clauses 38 to 45, wherein a distance between the second detector and the third detector along the adjustable headband is adjustable within a range of about 0.5 cm to about 2.5 cm.
47. The spectroscopy device of any of clauses 38 to 46, wherein the light source includes at least one light-emitting diode (LED).
48. The spectroscopy device of any of clauses 38 to 47, wherein the first detector, the second detector, and the third detector include at least one photodiode.
49. The spectroscopy device of any of clauses 38 to 48, further including: a circuit electrically coupled to the light source, the first detector, the second detector, and the third detector, the circuit being configured to: supply power to the light source; receive a first analog signal indicative of the first portion from the first detector; receive a second analog signal indicative of the second portion from the second detector; and receive a third analog signal indicative of the third portion from the first detector.
50. The spectroscopy device of clause 49, further including: a power source; and a switch configured to selectively connect the power source to the circuit.
51. The spectroscopy device of clause 50, wherein the power source includes a battery.
52. The spectroscopy device of any of clauses 49 to 51, further including: at least one analog-to-digital converter (ADC) configured to: convert the first analog signal into first data; convert the second analog signal into second data; and convert the third analog signal into third data.
53. The spectroscopy device of clause 52, further including: a transceiver configured to transmit a communication signal indicating at least one of the first data, the second data, or the third data.
54. The spectroscopy device of clause 52 or 53, further including: a processor configured to analyze the first data, the second data, and the third data.
55. The spectroscopy device of clause 54, further including: at least one output device physically coupled to the adjustable headband and configured to output an indication of at least one of the first data, the second data, or the third data.
56. The spectroscopy device of clause 55, wherein the at least one output device includes at least one of a display, a speaker, or a transceiver.
57. The spectroscopy device of any of clauses 38 to 56, further including: a watertight housing configured to at least partially enclose the light source, the first detector, the second detector, and the third detector.
58. A method, including: identifying first data indicative of a first portion of VNIR light detected by a first detector; identifying second data indicative of a second portion of the VNIR light detected by a second detector; identifying third data indicative of third portion of the VNIR light detected by a third detector; determining a metric indicative of a state of a cerebral structure based on the first data, the second data, and the third data; and outputting a signal based on the metric indicative of the state of the cerebral structure.
59. The method of clause 58, wherein identifying the first data includes detecting the first portion.
60. The method of clause 58 or 59, wherein identifying the second data includes detecting the second portion.
61. The method of any of clauses 58 to 60, wherein identifying the third data includes detecting the third portion.
62. The method of any of clauses 58 to 61, wherein the metric indicative of the state of the cerebral structure includes a regional oxygenation of blood traversing the cerebral structure.
63. The method of any of clauses 58 to 62, wherein determining the metric indicative of the state of the cerebral structure is further based on at least one of: an age of the subject; a weight of the subject; a head circumference of the subject; a skin pigmentation of the subject; a bilirubin state of the subject; an oxygen saturation of the subject; a partial pressure of carbon dioxide (pCO₂) in blood of the subject; a blood pressure of the subject; a skin thickness of the subject; a skull thickness of the subject; or a hematocrit of the subject.
64. The method of any of clauses 58 to 63, further including: determining that the metric indicative of the state of the cerebral structure is above an upper threshold or below a lower threshold; wherein outputting the signal based on the metric indicative of the state of the cerebral structure includes, in response to determining that the metric indicative of the state of the cerebral structure is above the upper threshold or below the lower threshold, outputting an alert.
65. The method of any of clauses 58 to 64, further including: determining that a change in the metric indicative of the state of the cerebral structure over a time interval is above an upper threshold or below a lower threshold; wherein outputting the signal based on the metric indicative of the state of cerebral structure includes, in response to determining that the change is above the upper threshold or below the lower threshold, outputting an alert.
66. A non-transitory computer readable medium storing instructions for performing the method of any of clauses 58 to 65.
67. A computing system configured to perform the method of any of clauses 58 to 65.
68. A system, comprising: at least one processor; and memory storing instructions that, when executed by the at least one processor, cause the at least one processor to perform operations comprising the method of any of clauses 58 to 65.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be used for realizing implementations of the disclosure in diverse forms thereof.

As will be understood by one of ordinary skill in the art, each implementation disclosed herein can comprise, consist essentially of or consist of its particular stated element, step, or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the implementation to the specified elements, steps, ingredients or components and to those that do not materially affect the implementation. As used herein, the term “based on” is equivalent to “based at least partly on,” unless otherwise specified.

Unless otherwise indicated, all numbers expressing quantities, properties, conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing implementations (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate implementations of the disclosure and does not pose a limitation on the scope of the disclosure. No language in the specification should be construed as indicating any non-claimed element essential to the practice of implementations of the disclosure.

Groupings of alternative elements or implementations disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

The complete disclosure of all patents, patent applications, and publications, and electronically available material cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern.

1. A cerebral spectroscopy monitoring device, comprising:
an adjustable headband configured to be disposed on an external surface of a head of a neonatal subject;
a light source physically coupled to the adjustable headband and configured to output visible and near infrared (VNIR) light through a skull of the neonatal subject;
a first detector physically coupled to the adjustable headband and configured to detect a first portion of the VNIR light scattered from and/or propagated through at least one of skin, muscle, or the skull of the neonatal subject;
a second detector physically coupled to the adjustable headband and configured to detect a second portion of the VNIR light scattered from and/or propagated through a brain tissue of the neonatal subject;
a third detector physically coupled to the adjustable headband and configured to detect a third portion of the VNIR light scattered from and/or propagated through a ventricle of the neonatal subject;
at least one output device configured to output an indication of a state of the brain tissue based on the first portion, the second portion, and third portion; and
a processor configured to:
detect an intraventricular hemorrhage of the neonatal subject by detecting a change in the third portion over time; and
in response to detecting the intraventricular hemorrhage, cause the at least one output device to output an alert.

2. The device of claim 1, wherein the neonatal subject is a preterm infant and/or is 72 hours old or less.

3. (canceled)

4. The device of claim 1, wherein the adjustable headband comprises:
an adjustable portion disposed;
between the light source and at least one of the first detector, the second detector, or the third detector;
between the first detector and the second detector; or
between the second detector and the third detector.

5-6. (canceled)

7. The device of claim 1, wherein the adjustable headband comprises an adjustable snap mechanism and/or an elastic material.

8. The device of claim 1, further comprising:
a biocompatible adhesive disposed on a surface of the adjustable headband, the biocompatible adhesive being configured to adhere the adjustable headband to the skin of the neonatal subject.

9. (canceled)

10. The device of claim 1, wherein the first portion is emitted from a tissue that is disposed below the surface of the skull of the neonatal subject by a depth of about 1 cm to about 2 cm;
wherein the second portion is emitted from a tissue that is disposed below the surface of the skull of the neonatal subject by a depth of about 1 cm to about 8 cm; and
wherein the third portion is emitted from a structure that is disposed below the surface of the skull of the neonatal subject by a depth of about 1.5 cm to about 10 cm.

11-17. (canceled)

18. The device of claim 1, wherein the indication of the state of the brain tissue comprises a regional blood oxygenation of blood traversing the brain tissue, and
wherein the processor is further configured to:
determine the regional blood oxygenation based on the second portion; and
correct the regional blood oxygenation based on the first portion and the third portion.

19. The device of claim 18, wherein the processor is configured to determine the regional blood oxygenation of the blood traversing the brain tissue further based on at least one of:
an age of the neonatal subject;
a weight of the neonatal subject;
a head circumference of the neonatal subject;
a skin pigmentation of the neonatal subject;
a bilirubin state of the neonatal subject;
an oxygen saturation of the neonatal subject;
a partial pressure of carbon dioxide (pCO₂) in blood of the neonatal subject;
a blood pressure of the neonatal subject;
a skin thickness of the neonatal subject;
a skull thickness of the neonatal subject; or
a hematocrit of the neonatal subject.

20. (canceled)

21. The device of claim 1, wherein the processor is further configured to determine a state of the ventricle by subtracting a magnitude of the first portion and a magnitude of the second portion from a magnitude of the third portion.

22-24. (canceled)

25. The device of claim 1, wherein the device further comprises:
a sensor configured to detect a physiological parameter of the neonatal subject, and
wherein the at least one output device is further configured to output an indication of the physiological parameter,
wherein the physiological parameter comprises a level of blood glucose, a hematocrit, a pulse, an EEG, a blood pressure, a pulse oxygenation, an airway parameter, an ECG, a blood flow, a pCO2, or a temperature.

26. (canceled)

27. A computing device, comprising:
at least one processor; and
memory storing instructions that, when executed by the at least one processor, cause the at least one processor to perform operations, comprising:
receiving first data indicative of a first portion of VNIR light detected by a first detector;
receiving second data indicative of a second portion of the VNIR light detected by a second detector;
receiving third data indicative of a third portion of the VNIR light detected by a third detector;
determining a metric indicative of a state of a brain tissue based on the first data, the second data, and the third data;
determining a metric indicative of a state of a ventricle based on the first data, the second data, and the third data; and
outputting a signal based on the metric indicative of the state of the brain tissue and/or the metric indicative of the state of the ventricle.

28. The computing device of claim 27, wherein the metric indicative of the state of the brain tissue comprises a regional oxygenation of the brain tissue.

29. The computing device of claim 27, wherein determining the metric indicative of the state of the brain tissue is further based on a secondary characteristic comprising at least one of:
an age of a subject;
a weight of the subject;
a head circumference of the subject;
a skin pigmentation of the subject;
a bilirubin state of the subject;
an oxygen saturation of the subject;
a partial pressure of carbon dioxide (pCO₂) in blood of the subject;
a blood pressure of the subject;
a skin thickness of the subject;
a skull thickness of the subject; or
a hematocrit of the subject.

30. (canceled)

31. The computing device of claim 27, wherein the operations further comprise:
determining that the metric indicative of the state of the brain tissue is above a first upper threshold or below a first lower threshold; and
determining that the metric indicative of the state of the ventricle is above a second upper threshold or below a second lower threshold,
wherein outputting the signal based on the metric indicative of the state of the brain tissue comprises, in response to determining that the metric indicative of the state of the brain tissue is above the first upper threshold or below the first lower threshold, outputting a first alert, and
wherein outputting the signal based on the metric indicative of the state of the ventricle comprises, in response to determining that the metric indicative of the state of the ventricle is above the second upper threshold or below the second lower threshold, outputting a second alert.

32. (canceled)

33. The computing device of claim 27, further comprising:
determining that a change in the metric indicative of the state of the brain tissue over a time interval or that a change in the metric indicative of the state of the ventricle over the time interval is above an upper threshold or below a lower threshold; and
outputting an alert.

34-57. (canceled)

58. A method, comprising:
identifying first data indicative of a first portion of VNIR light detected by a first detector;
identifying second data indicative of a second portion of the VNIR light detected by a second detector;
identifying third data indicative of third portion of the VNIR light detected by a third detector;
determining a metric indicative of a state of a cerebral structure based on the first data, the second data, and the third data; and
outputting a signal based on the metric indicative of the state of the cerebral structure.

59-61. (canceled)

62. The method of claim 58, wherein the metric indicative of the state of the cerebral structure comprises a regional oxygenation of blood traversing the cerebral structure.

63. (canceled)

64. The method of claim 58, further comprising:
determining that the metric indicative of the state of the cerebral structure is above an upper threshold or below a lower threshold;
wherein outputting the signal based on the metric indicative of the state of the cerebral structure comprises, in response to determining that the metric indicative of the state of the cerebral structure is above the upper threshold or below the lower threshold, outputting an alert.

65. The method of claim 58, further comprising:
determining that a change in the metric indicative of the state of the cerebral structure over a time interval is above an upper threshold or below a lower threshold;
wherein outputting the signal based on the metric indicative of the state of cerebral structure comprises, in response to determining that the change is above the upper threshold or below the lower threshold, outputting an alert.

66-67. (canceled)

68. The computing device of claim 27, wherein at least one of the first detector, the second detector, or the third detector is physically coupled to an adjustable headband.
